(19) United States
(12) Patent Application Publication
(10) Pub. No.: US 2022/0119897 A1
(43) Pub. Date: Apr. 21, 2022

(54) METHODS TO QUANTIFY VIRUS FROM WASTEWATER

(71) Applicant: The Research Foundation for the State University of New York, Syracuse, NY (US)

(72) Inventors: Hyatt C. GREEN, Syracuse, NY (US); Frank A. MIDDLETON, Fayetteville, NY (US)

(73) Assignee: The Research Foundation for the State University of New York, Syracuse, NY (US)

(21) Appl. No.: 17/348,636

(22) Filed: Jun. 15, 2021

Related U.S. Application Data
(60) Provisional application No. 63/039,338, filed on Jun. 15, 2020.

(51) Int. Cl. C12Q 1/70

(52) U.S. Cl. CPC C12Q 1/701; C12Q 1/705; C12Q 1/702; C12Q 1/70

(57) ABSTRACT

The present disclosure relates to a method of detecting and quantifying a virus-of-interest from human viral shed in a community, including: concentrating viral material from a wastewater sample to form a viral nucleic acid material; measuring an amount of a virus-of-interest nucleic acid in the viral nucleic acid material to obtain a first amount; measuring an amount of a marker or bacteriophage nucleic acid in the viral nucleic acid material to obtain a second amount; and forming a ratio of the first amount to the second amount to estimate a level of virus-of-interest from human viral shed within a community. In embodiments, methods for quantifying, monitoring, and/or identifying virus-of-interest such as SARS-CoV-2 are also disclosed.

### CROSS-REFERENCE TO RELATED APPLICATIONS

The present disclosure claims priority or the benefit under 35 U.S.C. § 119 of U.S. provisional application No. 63/039,338 filed Jun. 15, 2020, the contents of which are fully incorporated herein by reference.

### REFERENCE TO A SEQUENCE LISTING

This application contains a Sequence Listing in computer readable form, which is incorporated herein by reference.

### FIELD OF THE INVENTION

The present disclosure is in the fields of virology and epidemiology and includes methods for detecting virus-of-interest in a biological sample such as wastewater.

### BACKGROUND

Viruses continue to develop naturally resulting in new strains and diseases to human populations. For example, the World Health Organization (WHO) recently declared infection by the novel Severe Acute Respiratory Syndrome Coronavirus 2 (SARS-CoV-2), as a pandemic, and termed the related disease as coronavirus disease 2019 (COVID-19). Although a large percentage of persons infected with this novel virus experience mild to moderate respiratory, gastrointestinal, cardiovascular, or other discomforts without requiring medical care, infected persons with underlying medical problems, comorbidities, such as diabetes, cardiovascular disease, chronic respiratory disease or cancer are more likely to develop serious illness and/or die from COVID-19 or related secondary infections.

Transmission vectors of SARS-CoV-2, and variants thereof, are under heavy investigation at this time, and it is suspected the virus may be aerosolized and spread through saliva or discharge from the nose and throat when an infected person coughs or sneezes. While social distancing appears to reduce and/or contain the number of individuals infected at one time, disease transmission is problematic due to, among other things, asymptomatic infected individuals unwittingly transmitting and/or shedding virus in a community.

The inventors have noted reports that infected subjects, whether symptomatic or asymptomatic, shed virus and/or inactive viral particles thereof into community sewer systems through direct addition of fecal products or tissues used for collection of sinus drainage. While this presents an opportunity to investigate wastewater for incidence of disease, sampling and measuring wastewater for virus-of-interest such as e.g., SARS-CoV-2 and/or variants thereof is problematic due to low concentrations of virus or particles thereof alone, or in combination with contaminants in the wastewater. Wastewater including discharges or effluents from a sewer system typically include one or more substances not considered environmentally safe for direct discharge into other water systems. Such substances may include a plurality of ions, organics, biochemical reagents, heavy metals, heavy metal complexes, inorganic salts, inorganic reagents, and any other chemically or biologically active bodies such as waterborne pathogens, and other contaminants. Non-limiting examples of waterborne pathogens include bacterial, viral, fungal, and parasitic pathogens, such as fecal conforms. The mixture of contaminants and pathogens presents a difficult medium for viral DNA and RNA extraction therefrom, especially where concentrations of virus-of-interest are low.

The inventors have found that detection of a virus-of-interest and quantification thereof is also problematic considering the contaminant chemistry since large volumes of wastewater may need to be sampled for viral detection at lower limits. Large wastewater sample sizes are difficult to handle and process making sampling and high-volume analysis problematic.

The inventors have also observed that detecting and quantifying a virus-of-interest in wastewater does not provide enough information to determine the incidence of virus-of-interest disease or infection in an upstream community. More specifically, the determination that virus-of-interest is present may not alone correlate with the number of individuals infected in communities using the sewer system, or the particular strain or variant of SARS-CoV-2 that may be present in a community. The inventors have observed that markers are needed to link viral detection and quantification to incidence of viral infection or disease in a community.

What is needed are methods of detecting and/or quantifying virus-of-interest in wastewater, such as from a sewer system, and methods of recovering nucleic acids from virus-of-interest in wastewater. What is also needed are methods of concentrating and/or purifying viral nucleic acids from wastewater. Moreover, what is also needed are methods of surveilling wastewater to estimate or determine incidence of viral disease in a community.

### SUMMARY

The present disclosure relates to a combined set of methods for detecting and/or quantifying virus-of-interest in wastewater, such as from a sewer system, methods of recovering nucleic acids from virus-of-interest in wastewater, methods of concentrating and/or purifying viral nucleic acids from wastewater, and methods of surveilling wastewater to estimate or determine incidence of viral disease in a community. In embodiments, the virus-of-interest is SARS-CoV-2 and/or one or more variants of SARS-CoV-2.

In embodiments, the present disclosure relates to a method of detecting and quantifying a virus-of-interest from human viral shed in a community, including: concentrating viral material from a wastewater sample to form a viral nucleic acid material; measuring an amount of a virus-of-interest nucleic acid in the viral nucleic acid material to obtain a first amount; measuring an amount of a bacteriophage nucleic acid in the viral nucleic acid material to obtain a second amount; and forming a ratio of the first amount to the second amount to estimate a level of virus-of-interest from human viral shed within a community. In embodiments, the virus of interest is SARS-CoV-2 or a variant thereof.

In some embodiments, the present disclosure relates to a method of monitoring virus-of-interest human transmission in a community, including: (a) concentrating viral material from a wastewater sample to form a viral nucleic acid material; (b) measuring an amount of virus-of-interest nucleic acid in the viral nucleic acid material to obtain a first amount; (c) measuring an amount of bacteriophage nucleic acid in the viral nucleic acid material to obtain a second amount; (d) forming a ratio of the first amount to the second amount to estimate a level of virus-of-interest human viral shed within a community; and (e) repeating (a)-(d) in an amount sufficient to monitor virus-of-interest transmissions within a community. In embodiments, the viral material from a wastewater comprises or consists of SARS-CoV-2 or a variant thereof.

In some embodiments, a method of detecting and/or quantifying a virus-of-interest in wastewater, includes: concentrating viral material from a wastewater sample to form a viral nucleic acid material, wherein concentrating includes centrifuging the viral material through a sugar cushion in an ultracentrifuge; measuring an amount of virus-of-interest nucleic acid in the viral nucleic acid material to detect and/or quantify the virus-of-interest. In embodiments, the viral material from a wastewater sample is SARS-CoV-2 or a variant thereof.

In some embodiments, a method of detecting and/or quantifying a virus-of-interest in wastewater, includes: concentrating viral material from a wastewater sample to form a viral nucleic acid material, wherein concentrating includes centrifuging the viral material through a sugar cushion in an ultracentrifuge; measuring an amount of virus-of-interest nucleic acid in the viral nucleic acid material to detect and/or quantify the virus-of-interest, wherein the viral material is SARS-CoV-2 or a variant thereof.

In embodiments, the present disclosure relates to a method of determining the relative frequency of one or more viral strains in a wastewater sample. In embodiments, mixture modelling is performed to determine the relative frequency of one or more viral strains/isolates, such as one or more of SARS-CoV-2 or a variant thereof.

The illustrative aspects of the present disclosure are designed to solve the problems herein described and/or other problems not discussed.

### BRIEF DESCRIPTION OF THE DRAWINGS AND SEQUENCE LISTINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 is a flow diagram of a method of detecting and quantifying a virus-of-interest from human viral shed in a community in accordance with the present disclosure.

FIG. 2 is a flow diagram of a method of monitoring virus-of-interest human transmission in a community in accordance with the present disclosure.

FIG. 3 is a flow diagram of a method of detecting and/or quantifying a virus-of-interest in wastewater, in accordance with the present disclosure.

FIG. 4A is a photograph a raw wastewater above a sucrose solution prior to ultracentrifugation.

FIG. 4B is a photograph of a pellet produced by ultracentrifugation and residual debris on top of the 50% sucrose solution.

FIGS. 5A, 5B, and 5C are plots showing a relationship between crAssphage DNA load and population served (FIG. 5A), crAssphage RNA load and population served (FIG. 5B) and RNA load and crAssphage DNA load (FIG. 5C). Loads were calculated as the concentration x the flow rate.

FIGS. 6A and 6B are maps of incidence of viral cases by zip code (FIG. 7A) and SARS-CoV-2:crAssphage DNA ratios (FIG. 7B).

FIG. 7 depicts a map of treatment plants used in the analysis of the present disclosure.

FIG. 8 depicts the relationship between crAssphage nucleic acid concentration (copies per L) and daily influent flow at six Onondaga County access points. CrAssphage DNA concentration displays a significant negative relationship with influent flow (p less than 0.05 each site except 617 where p=0.052 crAssphage RNA concentration did not have a significant relationship with flows at any site.

FIGS. 9A, 9B, and 9C depict the relationship between crAssphage DNA load and population served (A), crAssphage RNA load and population served (B), and crAssphage RNA load and CrAssphage DNA load (C). Load is the product of nucleic acid concentration and flow rate.

FIG. 10 depicts the relationship between the log10(SARS-CoV-2):log 10(crAssphage) ratio (blue) and the number of new COVID-19 cases (red) in Cortland County, N.Y., US. Solid lines represent Loess smoothing functions of the data.

FIG. 11 depicts variation in per capita crAssphage nucleic acid loads between sites. Note that per capita loads are fairly consistent between sites.

FIG. 12 depicts association between smaller service areas and greater per capita crAssphage RNA load identified through conditional inference trees. Smaller service areas had higher crAssphage RNA loads.

FIG. 13 depicts association between SARS-CoV-2 classification from wastewater and the average daily number individuals to test positive for COVID-19 (per 10,000 people) (left) and the testing positivity rate (right) among people contributing to a sewershed in the seven days following sample collection.

FIG. 14 depicts an alignment of 69-70 mutation target region of S gene of SARS-CoV-2 Alpha variant and original Wuhan variant. Oligonucleotide target regions are indicated by red arrows or bars.

FIG. 15 depicts sensitivity and specificity of ESF_69/70 on synthetic DNA standards with and without the 69/70 deletion. The assay was tested for linearity on a serial dilution of a standard (5×10⁵, 5×10⁴, 5×10³, ×10², 5×10¹, and 5×10⁰ copies/reaction) containing the deletion. Assay specificity was demonstrated by the absence of amplification when 50,000 copies of a standard without the deletion (‘no del.’) was added as template.

FIGS. 16A depicts graphical clustering of wastewater derived samples following whole genome sequencing of SARS-CoV-2 with an optimized method. Sample information including PCR date, collection location and virus level is provided for representative samples that were found in a cluster of alpha/UK variant (B.1.1.7) sequences. FIG. 16B depicts the location of sequence variants identified in the wastewater samples displayed above, visualized in the SARS-CoV-2 genome.

FIG. 17A and 17B depict distribution of sequencing quality as a function of Ct value for detection of the SARS-CoV-2 virus using real-time qRT-PCR and the optimized sequencing method used for both saliva and wastewater analysis. Note that even up to 38 cycles, the likelihood of a high or moderate quality whole genome sequence is more than double that of a poor-quality genome sequence. Right, the amount of virus genome coverage after alignment compared with the Ct value for more than 1700 samples.

FIGS. 18 A and B depict SARS-CoV-2 genome sequence quality scores for wastewater RNA samples used to create next generation sequencing (NGS) libraries for variant mapping and strain identification. Across all samples, after index and adapter trimming the average phred quality score was 31.7.

FIG. 19 depicts sequence deconvolution of variants in wastewater composite samples. Results for one representative sample are shown. Where variants were observed, the majority of reads were used to identify the primary variant sequence present in the sample (top sequence in blue) and the minority of reads were used to identify the secondary variant sequence present (bottom sequence in orange). This sample is a Syracuse, N.Y. wastewater sample, obtained from an education institution. The overall proportion of each variant in the mapped reads is then estimated by multiplying the percent of alternate allele-specific reads and the percent of total allele-specific reads in that sample out of the total mapped reads.

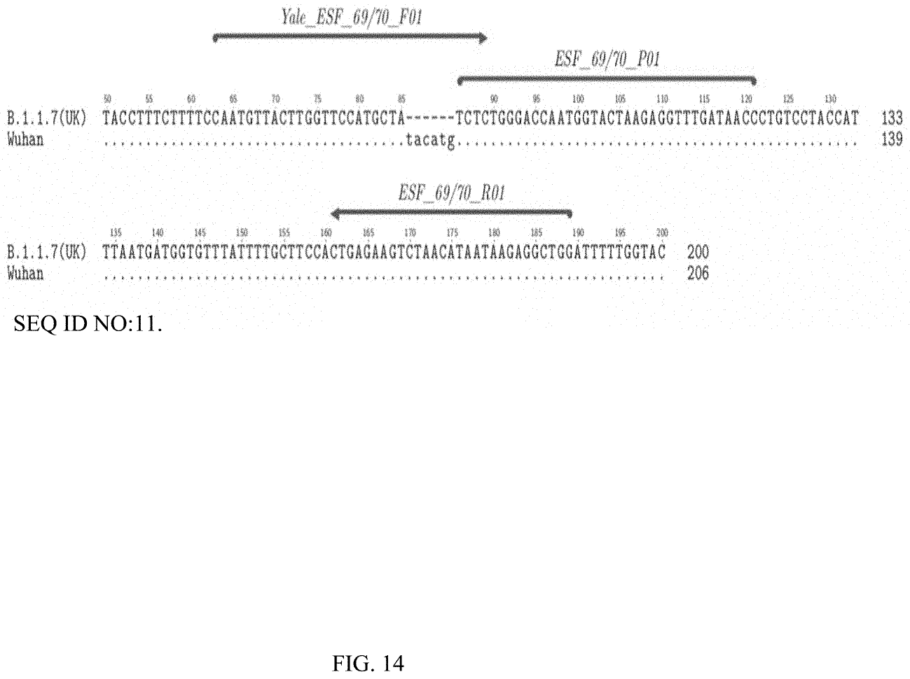

SEQ ID NO: 1 oligonucleotide sequence for nCoV_IP2-12669Fw.
SEQ ID NO: 2 oligonucleotide sequence for nCoV_IP2-12759Rv.
SEQ ID NO: 3 oligonucleotide sequence for nCoV_IP2-12696bProbe(+).
SEQ ID NO: 4 oligonucleotide sequence for nCoV_IP4-14059Fw.
SEQ ID NO: 5 oligonucleotide sequence for nCoV_IP4-14-14146Rv.
SEQ ID NO: 6 oligonucleotide sequence for nCoV_IP4-14084Probe(+).
SEQ ID NO: 7 oligonucleotide sequence for 056F1.
SEQ ID NO: 8 oligonucleotide sequence for 056R1.
SEQ ID NO: 9 oligonucleotide sequence for056P1.
SEQ ID NO: 10 is a primer for the 69-70 S deletion assay described below.
SEQ ID NO: 11 is a primer shown in Table 21 below.
SEQ ID NO: 12 is a primer shown in Table 21 below.
SEQ ID NO: 13 is depicted in FIG. 14.
SEQ ID NO: 14 is depicted in FIG. 14.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

The compositions and methods described herein include methods for detecting and/or quantifying virus-of-interest in wastewater, such as from a sewer system, methods of recovering nucleic acids from virus-of-interest in wastewater, methods of concentrating, enriching, and/or purifying viral nucleic acids from wastewater, and methods of surveilling wastewater to estimate or determine incidence of viral disease in a community. In some embodiments, the methods described herein advantageously: detect low levels of viral nucleic acid from wastewater such as wastewater having high levels of contamination; enable small volume sample collection, such as e.g, less than 50 mL of wastewater; and provide for a marker in wastewater useful in surveilling wastewater and estimating or determining incidence of viral disease in a community. Moreover, as SARS-CoV-2 variants continue to develop, the methods of the present disclosure are suitable for determining the relative frequency of one or more viral strains in a wastewater sample and/or performing mixture modelling to determine the relative frequency of one or more viral strains/isolates, such as one or more of SARS-CoV-2 or a variant thereof causing disease in an upstream geographical location. Performing the methods of the present disclosure is beneficial from an epidemiological perspective in that data generated may be used to form or alter containment or treatment strategies for communities and individuals therein.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s).

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

As used herein the term “cDNA” refers to a DNA molecule that can be prepared by reverse transcription from an RNA molecule obtained from a eukaryotic or prokaryotic cell, a virus, or from a sample solution in embodiments, cDNA lacks introns or intron sequences that may be present in corresponding genomic DNA. In embodiments, cDNA may refer to a nucleotide sequence that corresponds to the nucleotide sequence of an RNA from which it is derived. In embodiments, cDNA refers to a double-stranded DNA that is complementary to and derived from mRNA.

As used herein the “degree of identity” refers to the relatedness between two amino acid sequences or between two nucleotide sequences and is described by the parameter “identity”. In embodiments, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid or nucleotide in the two aligned sequences on a given position in the alignment; and 3) dividing the number of exact matches with the length of the reference sequence. In one embodiment, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid; or nucleotide in the two aligned sequences on a given position in the alignment; and 3) dividing the number of exact matches with the length of the longest of the two sequences. In some embodiments, the degree of sequence identity refers to and may be calculated as described under “Degree of Identity” in U.S. Pat. No. 10,531,672 starting at Column 11, line 56. U.S. Pat. No. 10,531,672 is incorporated by reference in its entirety. In embodiments, an alignment program suitable for calculating percent identity performs a global alignment program, which optimizes the alignment over the full-length of the sequences. In embodiments, the global alignment program is based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53). Examples of current programs performing global alignments using the Needleman-Wunsch algorithm are EMBOSS Needle and EMBOSS Stretcher programs, which are both available on the world wide web at www.ebi.ac.uk/Tools/psa/. In some embodiments a global alignment program uses the Needleman-Wunsch algorithm and the sequence identity is calculated by identifying the number of exact matches identified by the program divided by the “alignment length”, where the alignment length is the length of the entire alignment including gaps and overhanging parts of the sequences. In embodiments, the mafft alignment program is suitable for use herein.

The terms “deoxyribonucleotide” and “DNA” refer to a nucleotide or polynucleotide including at least one ribosyl moiety that has an H at the 2′ position of a ribosyl moiety. In embodiments, a deoxyribonucleotide is a nucleotide having an H at its 2′ position

The term “isolated” refers to a protein or DNA sequence that is removed from at least one component with which it is naturally associated.

By “hybridizable” or “complementary” or “substantially complementary” a nucleic acid (e.g. RNA, DNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a guide RNA, etc.): G can also base pair with U. For example, G/U base-pairing is partially responsible for the degeneracy (i.e., redundancy) of the genetic code in the context of tRNA anti-codon base-pairing with codons in mRNA. In embodiments, hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can include 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

An “isolated nucleic acid molecule” is a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

The term “nucleotide” refers to a ribonucleotide or a deoxyribonucleotide or modified form thereof, as well as an analog thereof.

As used herein, the term “nucleic acid molecule” refers to any molecule containing multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). As described further below, bases include C, T, U, C, and G, as well as variants thereof. As used herein, the term refers to ribonucleotides (including oligoribonucleotides (ORN)) as well as deoxyribonucleotides (including oligodeoxynucleotides (ODN)). The term shall also include polynucleosides (i.e., a polynucleotide minus the phosphate) and any other organic base containing polymer. Nucleic acid molecules can be obtained from existing nucleic acid sources (e.g., genomic or cDNA), but include synthetic (e.g., produced by oligonucleotide synthesis). In embodiments, the terms “nucleic acid” “nucleic acid molecule” and “polynucleotide” may be used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

In embodiments, the term “oligonucleotide” refers to a polynucleotide of between 4 and 100 nucleotides of single- or double-stranded nucleic acid (e.g., DNA, RNA, or a modified nucleic acid). However, for the purposes of this disclosure, there is no upper limit to the length of an oligonucleotide. Oligonucleotides are also known as “oligomers” or “oligos” and can be isolated from genes, transcribed (in vitro and/or in vivo), or chemically synthesized.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer including purine and pyrimidine bases or other natural, chemically or biochemically modified, non-natural, or derivatized nucleotide bases. The terms “polynucleotide” and “nucleic acid” should be understood to include, as applicable to the embodiments being described, single-stranded (such as sense or antisense) and double-stranded polynucleotides.

As used herein the term “prevent”, “preventing” and “prevention” of viral disease means (1) reducing the risk of a patient who is not experiencing symptoms of viral infection from developing viral disease, or (2) reducing the frequency of, the severity of, or a complete elimination of viral symptoms already being experienced by a subject.

As used herein, the term “virus-of-interest” refers to a virus or a portion of a virus subject to detecting, quantifying, concentrating, or monitoring in accordance with the present disclosure. In some embodiments, a virus-of-interest may include a Coronavirus such as SARS-CoV-2, SARS-CoV-2 variant, or a fragment thereof.

As used here, the term “SARS-CoV-2” refers to virus classified within the genus Betacoronavirus (subgenus Sarbecovirus) in the family Coronaviridae (subfamily Orthocoronavirinae), a family of single-strand positive-sense RNA viruses. In embodiments, the term “SARS-CoV-2” includes variants of SARS-CoV-2.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater. In embodiments, a component of interest includes a virus-of-interest, such as SARS-CoV-2 or a variant thereof.

“Substantially similar” refers to nucleic acid molecules wherein changes in one or more nucleotide bases result in substitution of one or more amino acids, but do not affect the functional properties of the protein encoded by the DNA sequence. “Substantially similar” also refers to nucleic acid molecules wherein changes in one or more nucleotide bases do not affect the ability of the nucleic acid molecule to mediate alteration of gene expression by antisense or co-suppression technology. “Substantially similar” also refers to modifications of the nucleic acid molecules of the instant disclosure (such as deletion or insertion of one or more nucleotide bases) that do not substantially affect the functional properties of the resulting transcript vis-a-vis the ability to mediate alteration of gene expression by antisense or co-suppression technology or alteration of the functional properties of the resulting protein molecule. The disclosure encompasses more than the specific exemplary sequences.

As used herein the term “treat”, “treating” and “treatment” of viral disease means reducing the frequency of symptoms of viral disease, eliminating the symptoms of viral disease, avoiding or arresting the development of symptoms of viral disease, ameliorating or curing an existing or undesirable symptom caused by viral disease, and/or reducing the severity of symptoms of viral disease.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before embodiments are further described, it is to be understood that this disclosure is not limited to particular embodiments described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### Description of Certain Embodiments

The present disclosure relates to a combined set of methods for detecting, enriching, and/or quantifying virus-of-interest in wastewater, such as from a sewer system. In embodiments, methods of the present disclosure include: recovering nucleic acids from virus-of-interest in wastewater; concentrating, enriching, and/or purifying or substantially purifying viral nucleic acids from wastewater; and surveilling wastewater to estimate or determine incidence of viral disease in a community. In embodiments, the virus-of-interest is SARS-CoV-2 and/or one or more variants of SARS-CoV-2. In embodiments, virus-of-interest is characterized as substantially purified by the methods of the present disclosure.

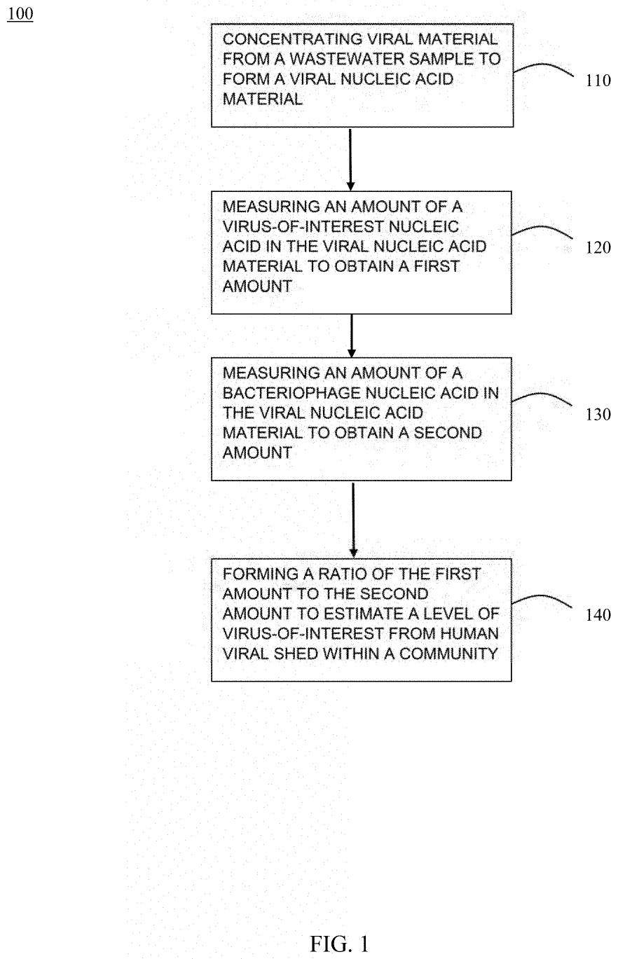

FIG. 1 is a flow diagram of a method 100 of detecting and quantifying a virus-of-interest from human viral shed in a community in accordance with the present disclosure. In embodiments, the virus-of-interest is one or more of an orthomyxovirus, influenza virus, influenza A, influenza B, an RNA virus, or a DNA virus. Non-limiting examples of RNA virus includes virus such as Reoviridae, Picornaviridae, Caliciviridae, Togaviridae, Arenaviridae, Retroviridae, Flaviviridae, Orthomyxoviridae, Paramyxoviridae, Bunyaviridae, Rhabdoviridae, Filoviridae, Coronaviridae, Astroviridae, or Bornaviridae. Non-limiting examples of Coronaviridae such as coronaviruses (CoVs) including SARS-CoV, MERS-CoV, SARS-CoV-2, SARS-CoV-2 variant, or any coronavirus that may cause disease in humans such as severe acute respiratory syndrome, or Middle East respiratory syndrome. Non-limiting examples of DNA virus includes Adenoviridae, Papovaviridae, Parvoviridae, Herpesviridae, Poxviridae, or Hepadnaviridae. In embodiments, the virus-of-interest is genus Flavivirus, Dengue virus (DENV), or a serotype thereof.

In embodiments, virus-of-interest includes SARS-CoV-2 and variants of SARS-CoV-2. In embodiments, the term SARS-CoV-2 includes virus from a reference strain of SARS-CoV-2 referred to as Wuhan-Hu-1 (GenBank accession MN908947) or ‘the original Wuhan strain’, sampled from a patient in Wuhan, China, on 26 Dec. 2019. As used herein the term SARS-CoV-2 includes variants of SARS-CoV-2. Although nomenclature for SARS-CoV-2 variants is not uniform one of ordinary skill in the art understands that established nomenclature for naming and tracking SARS-CoV-2 genetic lineages by GISAID, Nextstrain, and Pango are currently in use. Recently, the World Health Organization (WHO) recommended labeling SARS-CoV-2 variants using letters of the Greek Alphabet to refer to variants, such as variants of concern and variants of interest (See e.g., the world wide web at www.who.int/en/activities/tracking-SARS-CoV-2-variants/.). Accordingly, non-limiting examples of SARS-COV-2 variants include variants of concern, or variants having an observed increase in transmissibility or detrimental change in the COVID 19 epidemiology, increase in virulence or change in clinical presentation, or decrease in effectiveness of preventative measures such as social distancing and vaccination. Non-limiting examples of current variants-of-concern include the Alpha, Beta, Gamma, and Delta (WHO labelled variants), or B.1.17 (documented first in the U.K.), B.1.351 (documented first in South Africa), P.1 (documented first in Brazil), B.1.617.2 (documented first in India) (Pango lineage), respectively. Variants of interest include SARS-CoV-2 isolate, that when compared to a reference isolate, its genome has mutations with established or suspected phenotypic implications. Non-limiting examples of current variants of interest include Epsilon, Zeta, Eta, Theta, Iota, and Kappa (WHO labelled variants), or B.1427/B1.429, P2, B.1.525, P3, B,1.526, B.1.6171.1 (Pango lineage), respectively. Variants also include natural or manmade variants of SARS-CoV-2 that have not yet formed, thus are not yet identified or named.

In some embodiments, SARS-CoV-2 refers to Wuhan-Hu-1 (GenBank accession MN908947), sampled from a patient in Wuhan, China, on 26 Dec. 2019 (See e.g., Wu F, Zhao S, Yu B, Chen Y-M, Wang W, Song Z-G et al. A new coronavirus associated with human respiratory disease in China. Nature. 2020;579:265-9. doi: 10.1038/s41586-020-2008-3). That genome is 29 903 nucleotides (nt) in length and includes a gene order of similar structure to that seen in other coronaviruses: 5′-replicase ORF1ab-S-E-M-N-3′. The predicted replicase ORF1ab gene of Wuhan-Hu-1 is 21 291 nt in length. The ORF1ab polyprotein is predicted to be cleaved into 16 nonstructural proteins. ORF1ab is followed by a number of downstream open reading frames (ORFs). These include the predicted S (spike), ORF3a, E (envelope), M (membrane) and N (nucleocapsid) genes of lengths 3822, 828, 228, 669 and 1260 nt, respectively. Like SARS-CoV, Wuhan-Hu-1 also contains a predicted ORF8 gene (366 nt in length) located between the M and N genes. Finally, the 5′ and 3′ terminal sequences of Wuhan-Hu-1 are also typical of betacoronaviruses and have lengths of 265 nt and 229 nt, respectively. See e.g., Genomic sequencing of SARS-CoV-2: a guide to implementation for maximum impact on public health, 8 January 2021, COVID-19: Laboratory and diagnosis available on the world wide web at www.who. int/publications/i/item/9789240018440.

Referring now to FIG. 1, method 100 includes at process sequence 110 concentrating viral material from a wastewater sample to form a viral nucleic acid material. In embodiments, one or more wastewater samples, such as an aliquot of wastewater, is collected from one or more wastewater access points (e.g., wastewater treatment plants, influent pump stations, interceptor lines, outflow from an individual facility such as a school or dormitory). In embodiments, wastewater includes blackwater, greywater, and combinations thereof. In embodiments, samples may be stored at low temperature such as about 4° C., or about 2° C. to 7° C., following collection and/or transported on ice for processing at a later time, if needed. In embodiments, stored samples may be mixed to resuspend particulate matter therein and aliquoted. For example, samples may be mixed to resuspend particulate matter and aliquoted to an ultracentrifuge tube (such as e.g., ThermoFisher, #750000471 ultracentrifuge tube). In some embodiments, concentrating viral material may include centrifuging the viral material through a cushion in an ultracentrifuge, or in embodiments, centrifuging the viral material through a gradient in an ultracentrifuge. For example, a cushion may be positioned below a wastewater sample, such that the viral containing material will pass through the cushion or gradient upon centrifugation. In embodiments, suitable cushion for use herein includes dextran, colloidal silica such as used in PERCOLL™ brand density gradient medium, or sugar. Non-limiting examples of sugar includes monosaccharides, and disaccharides, such as sucrose. In embodiments, suitable cushion for use herein includes iodixanol. In some embodiments, suitable gradient for use herein includes two or more layers of dextran, colloidal silica such as used in in PERCOLL™ brand density gradient, or sugar. Non-limiting examples of sugar includes monosaccharides, and disaccharides, such as sucrose. In embodiments, suitable gradient for use herein includes two or more layers of iodixanol. In some embodiments, one or more gradient layers (such as 2, 3, 4, or several) are configured within an ultracentrifuge and the one or more gradient layers have a different density. Accordingly, in embodiments, a first gradient layer is provided having a first density, and a second gradient layer is provided having a second density different than the first density.

In some embodiments, concentrating viral material may include centrifuging the viral material through a sugar cushion in an ultracentrifuge, or in embodiments, centrifuging the viral material through a sugar gradient in an ultracentrifuge. In some embodiments, centrifuging the viral material through a sugar cushion in an ultracentrifuge further includes forming a solution including a buffer and sugar. In some embodiments, the solution includes a sugar such as sucrose in the amount of 40% to 60 percent weight of the total solution. In some embodiments, a sucrose cushion includes 40% to 60% sucrose, such as about 50% sucrose disposed within a buffer. In some embodiments, the sugar includes or consists of sucrose in an amount of about 50 percent weight of the total solution. In embodiments, the solution is provided below a wastewater or biological sample in a tube such as an ultracentrifuge tube.

In embodiments, suitable buffer may include e.g., THE buffer such as [20 mM Tris-HCl (pH 7.0), 100 mM NaCl, 2 mM EDTA]) added adjacent to or underneath the wastewater aliquot within a centrifuge tube to form one or more distinct layers. In some embodiments, pH value of the sugar, e.g., sucrose and buffer mixture or solution may be 5.5 to 8.5, or about 5.5, about 6.5, or about 7. In some embodiments, a sucrose gradient is formed including a first sugar layer including for example 40 to 60% sucrose, or about 50% sucrose combined with a buffer disposed atop or below a second sugar layer including for example 40 to 60% sucrose, or about 50% sucrose combined with a buffer, wherein the first layer and second layer include different amounts of sugar and the combination of layers forms a gradient within a centrifuge tube.

Still referring to process sequence 110 samples may be concentrated and/or purified by centrifugation. In embodiments centrifugation is performed at about 100,000 to 300,000×g below room temperature, such as e.g., 150,000×g at 4° C. In embodiments, the duration of the centrifugation may be between about 30 to 100 minutes such as 45 to 90 minutes. Non-limiting examples of an ultracentrifuge suitable for use herein include a Sorvall WX Ultra series with a Sorvall SURESPIN™ 630 rotor (ThermoFisher). In embodiments, supernatant may be decanted. In embodiments, viral nucleic acid material such as total viral nucleic acid material including DNA and RNA may form in pellets. Pellets containing viral nucleic acid material may be resuspended in liquid such as e.g., 200 μL 1× PBS. In some embodiments, viral nucleic acid material such as pellets may be stored at −20° C. for <12 hours prior to nucleic acid extraction.

Still referring to FIG. 1, method 100 includes at process sequence 120 measuring an amount of a virus-of-interest nucleic acid in the viral nucleic acid material to obtain a first amount. In embodiments, nucleic acid from the virus-of-interest may be quantified by known methods in the art. Non-limiting examples of quantitating virus include Reverse Transcriptase quantitative PCR (RT-qPCR), and methodology described in Institut Pasteur, Real-time RT-PCR assays for the detection of SARS-CoV-2, 2020 (herein incorporated by reference). A protocol for viral SARS-CoV-2 quantification including primers and process conditions is available on the world wide web at www.who. int/docs/default-source/coronaviruse/real-time-rt-per-assays-for-the-detection-of-sars-cov-2-institut-pasteur-paris.pdf?sfvrsn=3662fcb6_2. In accordance with this protocol, the materials, kits, primers, probes, nucleic acid extraction, conditions, mix preparations are all herein expressly incorporated by reference.

In embodiments, to detect a virus-of-interest such as SARS-CoV-2 or variants thereof, RT-qPCR may be used with a multiplex reaction containing the IP2 and IP4 assays (see e.g., Institut Pasteur, Real-time RT-PCR assays for the detection of SARS-CoV-2, 2020). In embodiments, reactions may include 6.25 uL Reliance One-Step Multiplex RT-qPCR Supermix, 0.4 μM each primer, 0.16 μM probes, molecular grade water, and 2.5 uL template total nucleic acids for a total reaction volume of 25 μL. In embodiments, PCR thermal cycling conditions may include 10 minutes at 50° C., 10 minutes at 95° C., followed by 45 cycles of 95° C. for 10 seconds and 59° C. for 30 seconds. In embodiments, a standard curve, including amplicons spanning the region of the SARS-CoV-2 virus interrogated by the IP2 and IP4 assays, ranging from 250 to 2.5 copies/reaction, may be used to convert Ct values to gene copies per reaction. Other known methods for quantifying the SARS-CoV-2 virus that have been used successfully on the wastewater samples by the inventors to provide quantitative information about SARS-CoV-2 levels include digital droplet PCR (ddPCR).

Still referring to FIG. 1, method 100 includes at process sequence 130 measuring an amount of a marker nucleic acid such as a bacteriophage nucleic acid in the viral nucleic acid material to obtain a second amount. In embodiments, the marker nucleic acid is from human gut derived virus or bacteria phage such from bacteria or virus abundant in the human gut. In embodiments, the marker is a bacteriophage such as ϕ6 (Phi 6) a bacteriophage of the virus family Cystoviridae, Escherichia virus MS2 bacteriophage, crAssphage, or combinations thereof. In embodiments, the bacteriophage is a biological marker such as a ubiquitous or common human gut bacterium. In embodiments, the bacteriophage suitable for use herein includes cross-assembly phage (crAssphage). In embodiments, measuring bacteriophage nucleic acid in the viral nucleic acid may be performed by any method known in the art such as e.g., quantitative PCR, or qPCR.

In embodiments, nucleic extraction and synthesis of cDNA may be employed to measure bacteriophage nucleic acid. For example, embodiment of the present disclosure includes nucleic acid extraction and synthesis of cross-assembly phage (crAssphage) cDNA. In embodiments, total nucleic acids may be extracted using known methods in the art including ALLPREP® POWERVIRAL® DNA/RNA Kit (Qiagen, Hilden, Germany) according to manufacturer's protocol. Samples may be eluted in RNase fee water such about 50 μL RNase free water. To assess the recovery of crAssphage RNA through the methods previously described, cDNA may be generated using the QuantiTect Reverse Transcription Kit (Qiagen) according to manufacturer's protocol.

In some embodiments, qPCR is used to detect the presence of crAssphage using the a CPQ_056 assay described in Stachler, et al, Quantitative CrAssphage PCR Assays for Human Fecal Pollution Measurement. Environmental science & 4technology 2017, 51, (16), 9146-9154 (herein entirely incorporated by reference). In embodiments, reactions may include 12.5 μL TaqMan® Environmental MasterMix (ThermoFisher), 1 μM primers, 80 nM probe, molecular grade water, and 2 μL template DNA for a total reaction volume of 25 μL. Each reaction may be run on either a QuantStudio3™ or QuantStudio5™ (ThermoFisher) under thermal cycling conditions including: 10 minutes at 95° C., followed by 40 cycles of 95° C. for 15 seconds and 60° C. for 1 minute. A DNA standard may be generated by purification of amplified PCR product using Roche High Pure PCR Template Preparation Kit. In embodiments, standard DNA quantity was assessed on NanoDrop spectrophotometer and Qubit® fluorometer. In embodiments, a standard curve, including purified amplicons ranging from 1×10⁶ to 5 copies/reaction, may be used to convert Ct values to gene copies per reaction.

Still referring to process sequence 130, in some embodiments, the CPQ_056 assay described above may be modified for SYBR Green chemistry. For example, SYBR Green qPCR may be used to assess recovery of bacteriophage such as crAssphage markers. In embodiments, reactions may include 12.5 μL TaqMan Environmental MasterMix, 1 μM CPQ_056 primers, 0.25 μL 10× SYBR Green dye, molecular grade water, and 2 μL template DNA for a total reaction volume of 25 μL. Thermal cycling conditions include 10 minute at 95° C., followed by 40 cycles of 95° C. for 15 seconds and 60° C. for 1 minute followed by a melt curve. In embodiments, a standard curve, including purified amplicons ranging from 1×10⁶ to 5 copies/reaction, may be used to convert Ct values to gene copies per reaction.

Still referring to FIG. 1, the method 100 includes at process sequence 140 forming a ratio of the first amount to the second amount to estimate a level of virus-of-interest from human viral shed within a community.

In some embodiments, in order to assess the proportion of viral nucleic acids remaining in each phase (including the pellet) after centrifugation, wastewater samples (2×20 ml) may be spiked with virus-of-interest such as SARS-CoV-2 or a variant thereof (resulting in approximately 580 gene copies per ml wastewater). For example, pellets may be generated by ultracentrifugation at 150,000×g for 45 minutes and the following layers were removed; aqueous upper (top 10 ml), aqueous lower (second 9 ml), cushion interface (1.5 ml, specifically targeting visible suspended particles on top of sucrose layer), sucrose upper (6 ml), sucrose lower (6 ml), and pellet (appx. 200 μL). In embodiments, an aliquot such as a 200 μL aliquot of each layer may be extracted using the PowerViral kit (Qiagen) and extracts analyzed with the IP2IP4 assay in 25 μL reaction volumes under the conditions described previously.

In some embodiments, process 100 may be performed in a duration between 1-5 hours, 2-5 hours, 3-5 hours, less than 5 hours, less than 4 hours, or less than 3 hours from the time a wastewater sample is collected or from the time the process sequence 100 commences.

In some embodiments, correlating evidence of virus-of-interest to disease transmission in wastewater may be performed by retrieving a total number of known viral disease (caused by the virus-of-interest) in a known geographical area such as a wastewater catchment area. In embodiments, a standardized number of incidence of disease caused by the virus-of-interest is generated to calculate cumulative incidence per 1,000 residents. Cumulative incidence may be used to form a ratio of virus-of-interest to the marker such as a bacteriophage. For example, in embodiments, standardizing the number of COVID-19 cases may be performed using one or more predetermined geographical areas, such as by ZIP code to the ZIP code population to calculate a cumulative incidence per 1,000 residents. This may provide a visualization of the cumulative incidence alongside the ratio of log₁₀(SARS-CoV-2): log₁₀(crAssphage).

In some embodiments, non-limiting examples of variations of the ratio of the present disclosure includes, log 10(SARS-CoV-2:crAssphage) and variations thereof useful at predicting community transmission such as e.g., either crAssphage cDNA or crAssphage DNA. In some embodiments, the following non-limiting values may be important for evaluating community spread: log 10(SARS-CoV-2): log 10(crAssphage DNA): 0.14 to 0.40; log 10(SARS-CoV-2): log 10(crAssphage cDNA): 0.3 to 0.75; log 10(SARS-CoV-2:crAssphage DNA): −4.7 to −2.5; log 10(SARS-CoV-2:crAssphage cDNA): −2.0 to −0.4. In some embodiments, the following relationship and ranges of values may be predictive of transmission: SARS-CoV-2 copies/ml/(log 10(crAssphage cDNA): log 10(crAssphage DNA)): 10 to 65 copies/ml or higher. In some embodiments, the latter may more accurately account for decay of viruses in the wastewater infrastructure.

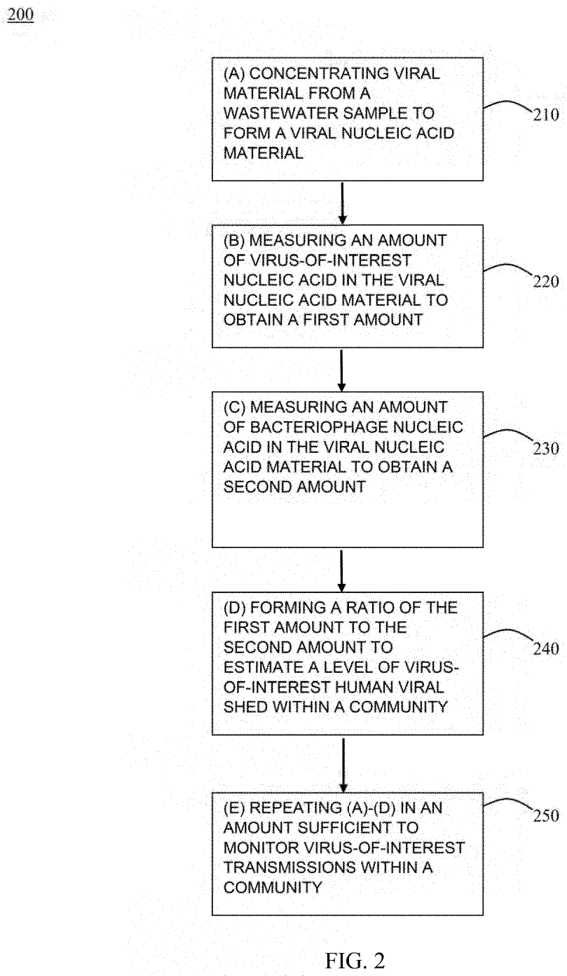

Referring now to FIG. 2, a flow diagram of a method of monitoring virus-of-interest human transmission in a community in accordance with the present disclosure. In embodiments, method 200 includes at process sequence 210(a) concentrating viral material from a wastewater sample to form a viral nucleic acid material. In embodiments, concentrating viral material may be performed as described above. For example, in some embodiments, concentrating viral material may include centrifuging the viral material through a cushion such as a sugar cushion in an ultracentrifuge, or in embodiments, centrifuging the viral material through a gradient such as a sugar gradient in an ultracentrifuge. In some embodiments, centrifuging such as ultra-centrifuging includes and forming a solution including a buffer and sugar and passing the viral material through a sugar cushion in an ultracentrifuge. In embodiments, the solution includes a sugar in the amount of 40 to 60 percent weight of the total solution. In embodiments, the sugar includes sucrose in an amount of about 50 percent weight of the total solution. In embodiments, the virus-of-interest is SARS-CoV-2.

Referring now to FIG. 2, method 200 at process sequence 220 includes measuring an amount of virus-of-interest nucleic acid in the viral nucleic acid material to obtain a first amount. In embodiments, measuring an amount of virus-of-interest nucleic acid may be performed as described above. For example, RT-qPCR may be used to quantitate virus-of-interest such as SARS-CoV-2.

Still referring to FIG. 2, method 200 at process sequence 230 includes measuring an amount of bacteriophage nucleic acid in the viral nucleic acid material to obtain a second amount. In embodiments, the second amount may be determined as described above or in method known in the art. In embodiments, qPCR is used to detect the presence of a bacteriophage such as crAssphage using the a CPQ_056 assay described in Stachler, et al, Quantitative CrAssphage PCR Assays for Human Fecal Pollution Measurement. Environmental science & technology 2017, 51, (16), 9146-9154.

Still referring to FIG. 2, method 200 at process sequence 240 includes forming a ratio of the first amount to the second amount to estimate a level of virus-of-interest human viral shed within a community. In embodiments, the ratio is performed by method known in the art or described above. In embodiments, when the virus of interest is SARS-CoV-2, a ratio may be formed such as log₁₀(SARS-CoV-2): log₁₀(crAssphage) and used to estimate the level of SARS-CoV-2 shed within a community. Other ratios such as those described above are suitable for use herein.

Still referring to FIG. 2, method 200 at process sequence 250, the process sequences (a)-(d) of method 200 may be repeated in an amount sufficient to monitor virus-of-interest transmissions within a community. For example, process sequences (a)-(d) may be repeated every day for 1 month to monitor virus-of-interest in a community, every day for 2 months to monitor virus-of-interest in a community, or every day for 3-12 months to monitor virus-of-interest in a community. The number of repetitions and intervals therebetween may be varied or predetermined. For example, process sequences (a)-(d) may be repeated once a week for month(s) to monitor virus-of-interest in a community. The information obtained from monitoring may be used to alter or reconsider containment strategies and treatments for individuals in need thereof.

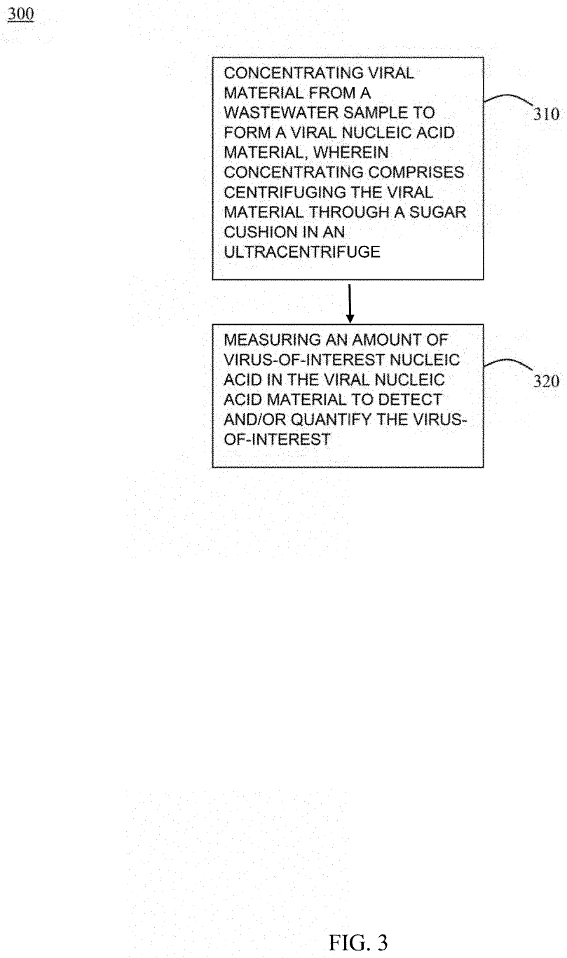

Referring now to FIG. 3, a flow diagram of a method of detecting and/or quantifying a virus-of-interest in wastewater is shown in accordance with the present disclosure. In embodiments, method 300 includes at process sequence 310 concentrating viral material from a wastewater sample to form a viral nucleic acid material, wherein concentrating includes centrifuging the viral material through a cushion such as a sugar cushion in an ultracentrifuge. In embodiments, concentrating viral material may be performed as described above. For example, in some embodiments, concentrating viral material may include centrifuging the viral material through a cushion such as a sugar cushion in an ultracentrifuge, or in embodiments, centrifuging the viral material through a gradient such as a sugar gradient in an ultracentrifuge. In some embodiments, centrifuging such as ultra-centrifuging includes forming a solution including a buffer and sugar and passing the viral material through a sugar cushion in an ultracentrifuge. In embodiments, the solution includes a sugar in the amount of 40 to 60 percent weight of the total solution. In embodiments, the sugar includes sucrose in an amount of about 50 percent weight of the total solution. In embodiments, the virus-of-interest is SARS-CoV-2.

In embodiments, method 300 includes at process sequence 320 measuring an amount of virus-of-interest nucleic acid in the viral nucleic acid material to detect and/or quantify the virus-of-interest. For example, in embodiments, nucleic acid from the virus-of-interest may be quantified by methods described above such as Reverse Transcriptase quantitative PCR (RT-qPCR), and methodology such as described in Institut Pasteur, Real-time RT-PCR assays for the detection of SARS-CoV-2, 2020 (herein incorporated by reference).

In embodiments, the present disclosure relates to a method of detecting and quantifying a virus-of-interest from human viral shed in a community, including: concentrating viral material from a wastewater sample to form a viral nucleic acid material; measuring an amount of a virus-of-interest nucleic acid in the viral nucleic acid material to obtain a first amount; measuring an amount of a marker or bacteriophage nucleic acid in the viral nucleic acid material to obtain a second amount; and forming a ratio of the first amount to the second amount to estimate a level of virus-of-interest from human viral shed within a community. In some embodiments, the virus-of-interest is an orthomyxovirus, influenza virus, influenza A, influenza B, an RNA virus, or a DNA virus. In some embodiments, the RNA virus is characterized as one of Reoviridae, Picornaviridae, Caliciviridae, Togaviridae, Arenaviridae, Retroviridae, Flaviviridae, Orthomyxoviridae, Paramyxoviridae, Bunyaviridae, Rhabdoviridae, Filoviridae, Coronaviridae, Astroviridae, or Bornaviridae. In some embodiments, the DNA virus is characterized as Adenoviridae, Papovaviridae, Parvoviridae, Herpesviridae, Poxviridae, or Hepadnaviridae. In some embodiments, the virus-of-interest is SARS-CoV-2 or a variant thereof. In some embodiments, the virus-of-interest is a Dengue virus. In some embodiments, the bacteriophage nucleic acid is cross-assembly phage nucleic acid. In some embodiments, concentrating viral material further includes centrifuging the viral material through a cushion such as a sugar cushion in an ultracentrifuge. In some embodiments, concentrating viral material further includes centrifuging the viral material through a gradient such as a sugar gradient in an ultracentrifuge. In some embodiments, centrifuging the viral material through a sugar cushion in an ultracentrifuge further includes forming a solution including a buffer and sugar. In some embodiments, the solution includes a sugar in the amount of 40 to 60 percent weight of the total solution. In some embodiments, the sugar includes or consists of sucrose in an amount of about 50 percent weight of the total solution. In some embodiments, the viral material includes inactivated or fragmented virus. In some embodiments, concentrating viral material from a wastewater sample to form a viral nucleic acid material is performed under conditions to purify the viral material. In embodiments, viral material is substantially purified. In some embodiments, measuring the amount of virus-of-interest nucleic acid and bacteriophage nucleic acid is performed by qPCR analysis including one or more fluorescent materials.

In some embodiments, the present disclosure relates to a method of monitoring a virus-of-interest human transmission in a community, including: (a) concentrating viral material from a wastewater sample to form a viral nucleic acid material; (b) measuring an amount of virus-of-interest nucleic acid in the viral nucleic acid material to obtain a first amount; (c) measuring an amount of marker or bacteriophage nucleic acid in the viral nucleic acid material to obtain a second amount; (d) forming a ratio of the first amount to the second amount to estimate a level of virus-of-interest human viral shed within a community; and (e) repeating (a)-(d) in an amount sufficient to monitor virus-of-interest transmissions within a community. In embodiments, concentrating viral material further includes centrifuging the viral material through a sugar cushion in an ultracentrifuge. In some embodiments, centrifuging the viral material through a sugar cushion in an ultracentrifuge further includes forming a solution comprising a buffer and sugar. In some embodiments, the solution includes a sugar in the amount of 40 to 60 percent weight of the total gradient-forming solution. In some embodiments, the sugar includes sucrose in an amount of about 50 percent weight of the total solution. In some embodiments, the virus-of-interest is SARS-CoV-2.

In some embodiments, the present disclosure relates to a method of detecting and/or quantifying a virus-of-interest in wastewater, including: concentrating viral material from a wastewater sample to form a viral nucleic acid material, wherein concentrating includes centrifuging the viral material through a cushion such as a sugar cushion in an ultracentrifuge; and measuring an amount of virus-of-interest nucleic acid in the viral nucleic acid material to detect and/or quantify the virus-of-interest. In embodiments, centrifuging the viral material through a cushion such as a sugar cushion in an ultracentrifuge further includes forming a solution including a buffer and sugar. In some embodiments, the solution includes a sugar in the amount of 40 to 60 percent weight of the total solution. In some embodiments, the sugar includes sucrose in an amount of about 50 percent weight of the total solution. In some embodiments, the wastewater is characterized as an aliquot of less than 50 mL of raw wastewater, or less than 25 mL of raw wastewater, or between 1 to 25 mL of raw wastewater, or about 15 to about 20 mL of wastewater. In some embodiments, concentrating viral material includes purifying or substantially purifying the nucleic acid of the viral material. In some embodiments, the virus-of-interest is SARS-CoV-2 or a variant thereof.

In some embodiments, the present disclosure relates to a method of detecting and quantifying a virus-of-interest from human viral shed in a community, including: concentrating viral material from a wastewater sample to form a viral nucleic acid material; measuring an amount of a virus-of-interest nucleic acid in the viral nucleic acid material to obtain a first amount; measuring an amount of a marker nucleic acid in the viral nucleic acid material to obtain a second amount; and forming a ratio of the first amount to the second amount to estimate a level of virus-of-interest from human viral shed within a community. In embodiments, the marker nucleic acid is from human gut derived virus or bacteriophage such from bacteria or virus abundant in the human gut. In embodiments, the marker is a bacteriophage such as ϕ6 (Phi 6) a bacteriophage of the virus family Cystoviridae, Escherichia virus MS2 bacteriophage, crAssphage, or combinations thereof.

In some embodiments, a method of detecting and/or quantifying a virus-of-interest in wastewater, includes: concentrating viral material from a wastewater sample to form a viral nucleic acid material, or substantially purse nucleic acid material, wherein concentrating includes centrifuging the viral material through a cushion or gradient in an ultracentrifuge; measuring an amount of virus-of-interest nucleic acid in the viral nucleic acid material to detect and/or quantify the virus-of-interest. In embodiments, suitable cushion for use herein includes dextran, colloidal silica such as used in PERCOLL™ brand gradient, or sugar. Non-limiting examples of sugar includes monosaccharides, disaccharides, such as sucrose. In embodiments, suitable cushion for use herein includes iodixanol. In some embodiments, suitable gradient for use herein includes two or more layers of dextran, colloidal silica such as used in PERCOLL™ brand gradient, or sugar. Non-limiting examples of sugar for use in a gradient includes monosaccharides, disaccharides, such as sucrose. In embodiments, suitable gradient for use herein includes two or more layers of iodixanol. In some embodiments, the gradient layers are configured within a centrifuge two and the gradient layers have a different density.

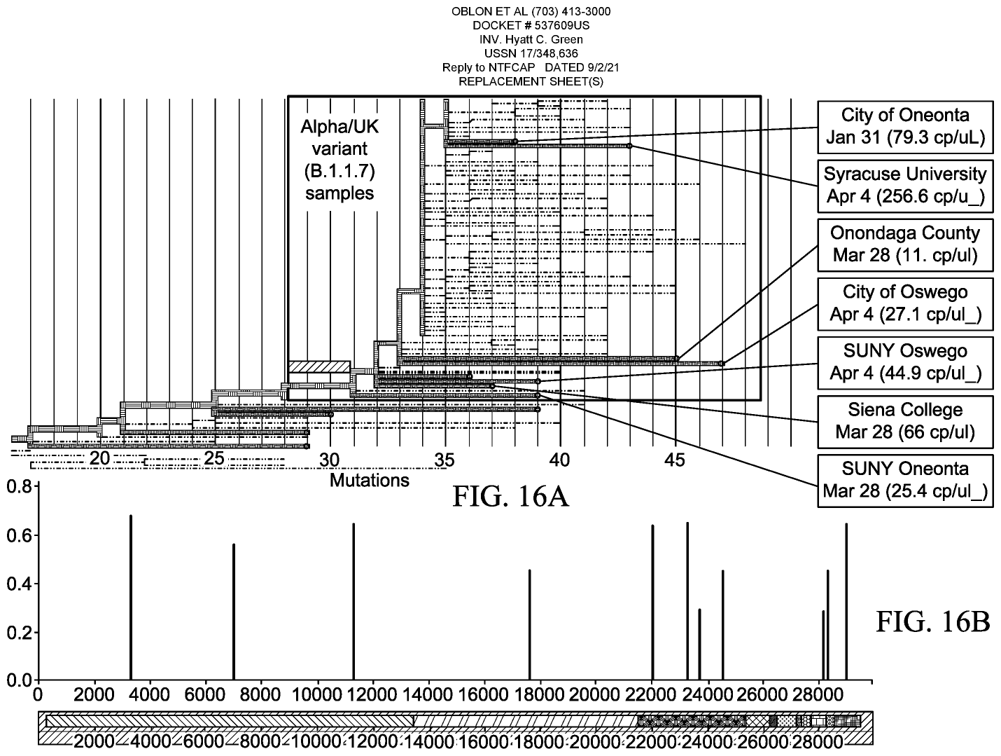

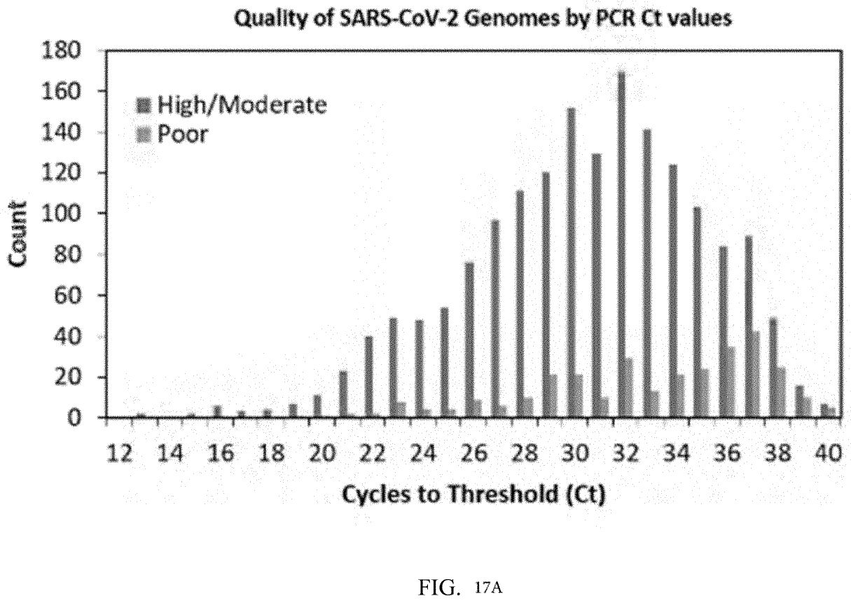

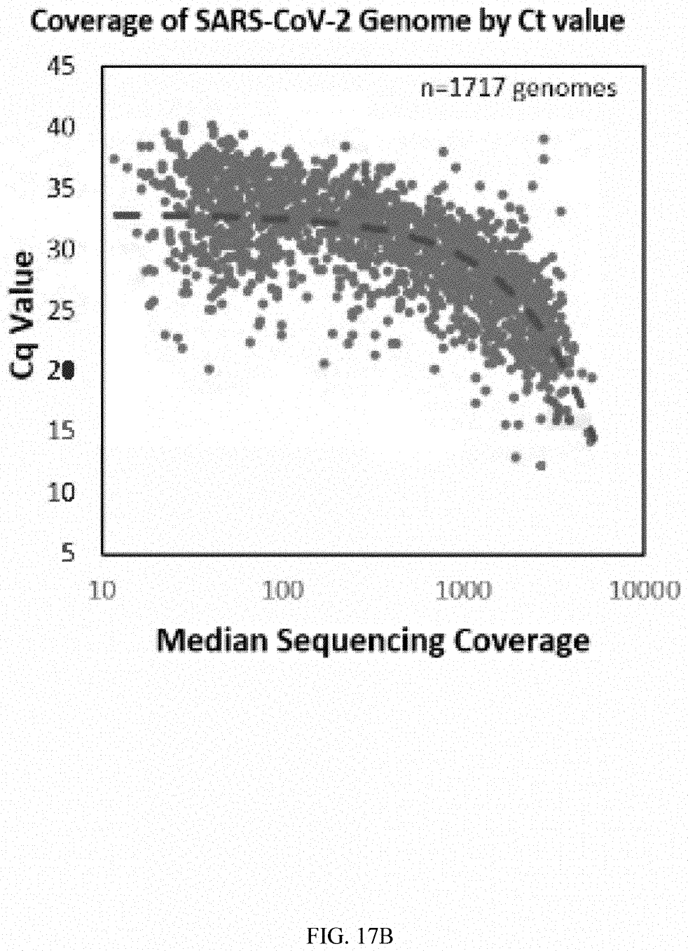

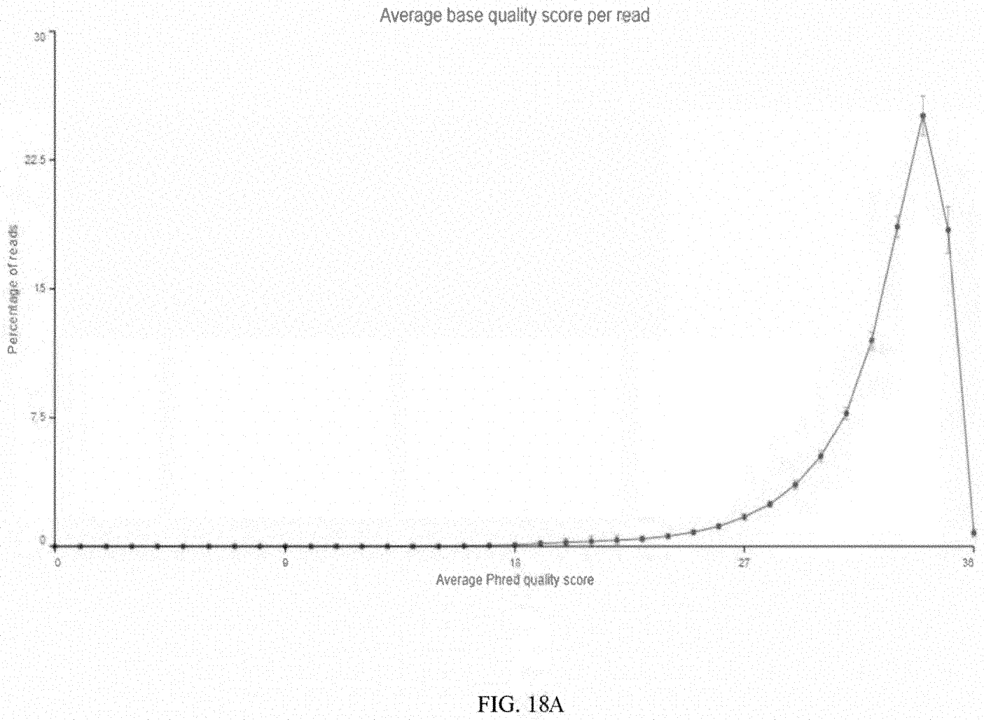

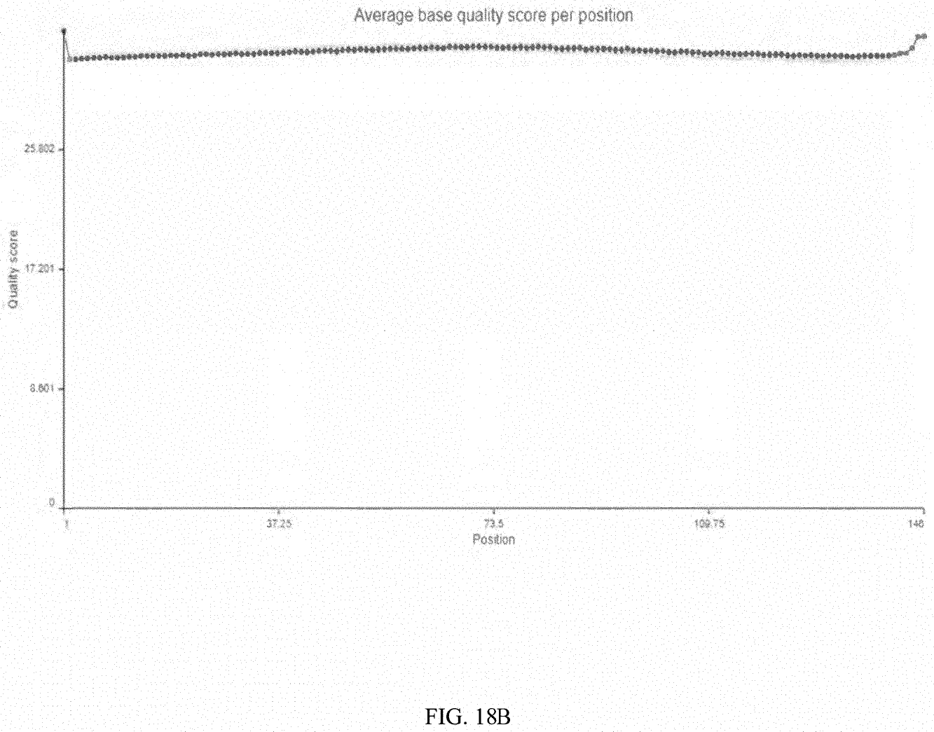

In some embodiments, the present disclosure includes a method of determining the relative frequency of one or more viral strains is a wastewater sample. In embodiments, mixture modelling is performed to determine the relative frequency of one or more viral strains, such as one or more of SARS-CoV-2 or a variant thereof. See, e.g. example IV below. In embodiments, the methods of the present disclosure include: monitoring for one or more nucleotide changes in a virus-of-interest. In embodiments, the methods include monitoring for one or more specific or individual nucleotide changes; and generating whole genome sequence data for wastewater samples. In embodiments, a suitable sequencing protocol includes a sequencing protocol modified from the ARCTICv3 method, that achieves whole-genome viral sequence coverage sufficient to clearly identify variant lineages (FIG. 16), even when the Ct value for detection of the virus exceeds 35 cycles (FIG. 17). See e.g., example IV below.

### EXAMPLES

### Example I

Materials and Methods—(The methods include those described in Green, et al., Quantification of SARS-CoV-2 and cross-assembly phage (crAssphage) from wastewater to monitor coronavirus transmission within communities (herein incorporate by reference in its entirety). This reference can be found on the world wide web at www.medrxiv.org/content/10.1101/2020.05.21.20109181v1.

### Wastewater Ultracentrifugation

Twenty-four-hour composite wastewater samples (1.9 L) were collected from 11 access points (i.e., wastewater treatment plants, influent pump stations, or interceptor lines) in Syracuse, N.Y. and other locations in Onondaga County, N.Y. on May 6 and May 13, 2020 (Table 1).

TABLE 1
Facility Characteristics and average flow (millions of gallons per day)
on May 6 and May 13, 2020.
Table 1:

| | | | Flow | Flow |
|---|---|---|---|---|
| Facility/ | | Population | May 6, 2020 | May 13,2020 |
| Catchment ID | Facility Type | Served | (M.G.D.) | (M.G.D.) |
| 600 | Contributor to | 3,841 | 1.25 | 0.95 |
| | 604 | | | |
| 601 | WWTP | 25,300 | 5 | 4.5 |
| 603 | Contributor to | 47,688 | 11.16 | 9.36 |
| | 604 | | | |
| 604 | WWTP | 223,900 | 70.3 | 62.2 |
| 605 | WWTP | 25,600 | 4.1 | 4.7 |
| 606 | WWTP | 37,800 | 5.4 | 4.9 |
| 610 | Contributor to | 112,747 | 29.1 | 25.7 |
| | 604 | | | |
| 617 | WWTP | 20,500 | 3.4 | 3.5 |
| 619 | WWTP | 12,800 | 1.9 | 1.7 |
| 725 | Contributor to | 39,262 | 11.2 | 10.01 |
| | 604 | | | |
| 1700 | Contributor to | 20,347 | 17.58 | 16.16 |
| | 604 | | | |

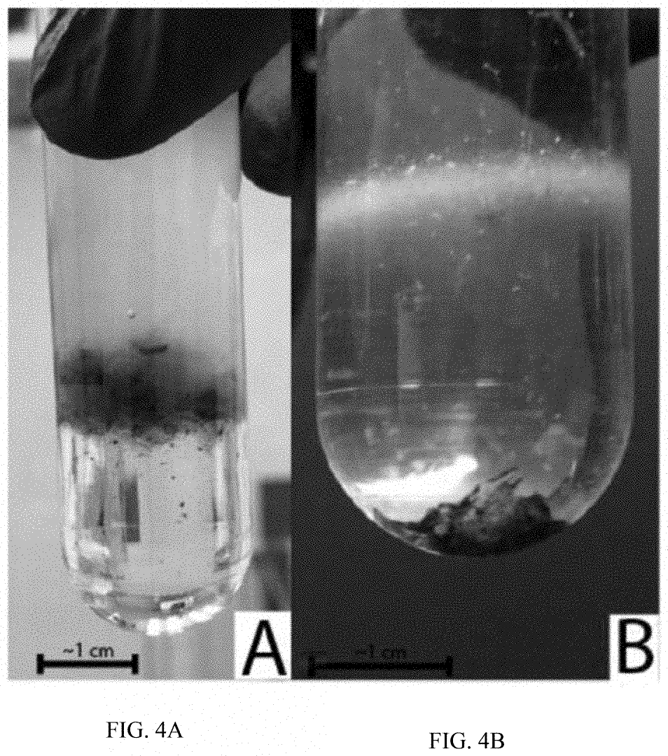

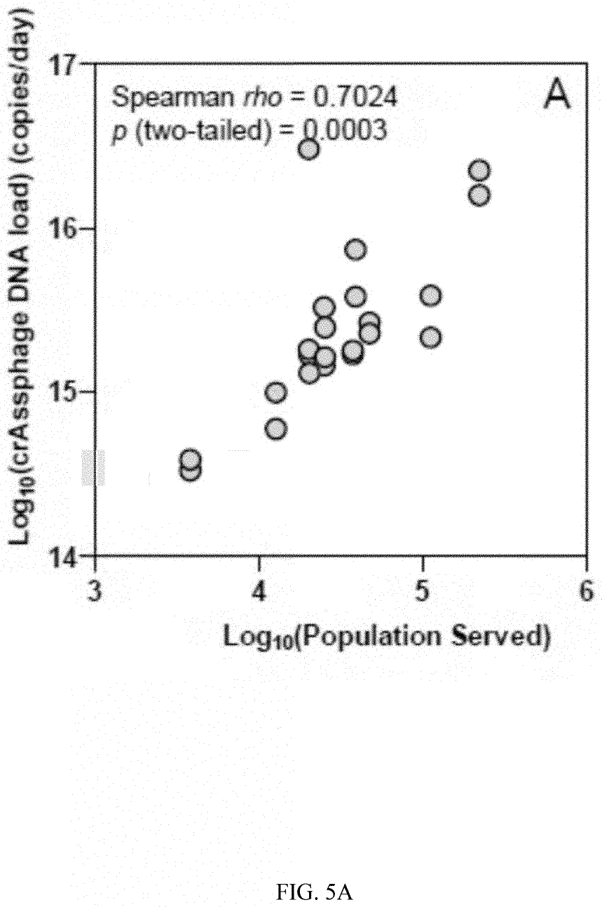

Samples were stored at 4° C. following collection and transported on ice to Upstate Medical University (Syracuse, N.Y.) for processing the next morning. Upon receipt, samples were mixed to resuspend particulates and 20 ml was aliquoted to a 38.5 ml ultracentrifuge tube (e.g., ThermoFisher, #750000471 brand ultracentrifuge tube). A 12 ml sucrose cushion (50% sucrose in THE buffer [20 mM Tris-HCl (pH 7.0), 100 mM NaCl, 2 mM EDTA]) was carefully added underneath the wastewater with a 10 ml disposable serological pipette so that two distinct layers were formed (FIG. 4A). See e.g., a first layer, e.g., a cloudy layer, disposed atop a second layer (clear) in FIG. 4A. Fifty percent sucrose yielded higher crAssphage DNA concentrations than 20% or 70% (See Table 2).

TABLE 2

| | | | Mean DNA Copies crAssphage/ |
|---|---|---|---|
| Sucrose | Spin Time | | mL Initial WW Source |
| Concentration | (Minutes) | Replicate | (+/−SD) |
| 20% | 20 | 1 | 1.95E4 (4.40E2) |
| 20% | 20 | 2 | 2.53E4 (5.95E2) |
| 50% | 90 | 1 | 9.04E4 (3.56E3) |
| 50% | 90 | 2 | 1.27E5 (1.88E3) |
| 75% | 150 | 1 | 3.26E4 (3.41E2) |
| 75% | 150 | 2 | 7.52E4 (8.55E2) |

In batches of six, samples were then purified by centrifugation at 150,000×g at 4° C. for either 90 minutes (May 6) or 45 minutes (May 13) on a Sorvall WX Ultra series with a Sorvall SURESPIN™ 630 rotor (ThermoFisher). Centrifugation times of 45 and 90 minutes provided roughly the same recovery of viral nucleic acids (See Table 3 below).

TABLE 3

| | | Mean DNA Copies crAssphage/ | Mean cDNA Copies crAssphage/ |
|---|---|---|---|
| Spin Time | | mL Initial WW Source | mL Initial WW Source |
| (Minutes) | Replicate | (+/−SD) | (+/−SD) |
| 30 | 1 | 6.74E4 (1.57E3) | 1.40E1 (3.68E0) |
| 30 | 2 | 7.21E4 (4.47E3) | <LOQ |
| 45 | 1 | 9.89E4 (4.16E3) | 3.46E1 (6.72E0) |
| 45 | 2 | 9.90E4 (2.82E3) | 3.42E1 (1.36E1) |
| 75 | 1 | 9.39E4 (5.86E3) | <LOQ |
| 75 | 2 | 1.19E5 (3.80E3) | 1.14E1 (5.77E0) |

Supernatant was then decanted, and pellets (FIG. 4B) were resuspended in 200 μL 1× PBS. Pellets were stored at -20° C. for <12 hours prior to nucleic acid extraction.

Distilled water (20 ml) was used as a processing blank.
Nucleic Extraction and Synthesis of CrAssphage cDNA

Total nucleic acids were extracted using the AlIPrep® PowerViral® DNA/RNA Kit (Qiagen, Hilden, Germany) according to manufacturer's protocol. Samples were eluted in 50 μL RNase free water. Extraction blanks using distilled water were performed in each extraction batch to assess contamination. To assess the recovery of crAssphage RNA through the methods previously described, cDNA was generated using the QuantiTect Reverse Transcription Kit (Qiagen) according to manufacturer's protocol.

CrAssphage qPCR

qPCR was used to detect the presence of crAssphage using the previously developed CPQ_056 assay (See Table 4 below).

TABLE 4
qPCR ASSAY USED

| ASSAY | | OLIGONUCLEOTIDE | OLIGONUCLEOTIDE | Amplicon | |
|---|---|---|---|---|---|
| NAME | TARGET | NAME | SEQUENCE (5′-3′) | Length | Reference |
| IP21 | SARS- | nCoV_IP2-12669Fw | ATGAGCTTAGTCCTGTTG | 108 bp | 12-Institute |
| P4 | CoV-2 | | (SEQ ID NO: 1) | | Pasteur, |
| Multi | | nCoV_IP2-12759Rv | CTCCCTTTGTTGTGTTGT | | Real-time |
| plex | | | (SEQ ID NO: 2) | | RT-PCT |
| | | nCoV_IP2- | [5′]Hex | | Assays for |
| | | 12696bProbe(+) | AGATGTCTT[Zen]GTGC | | detection |
| | | | TGCCG | | of SARS- |
| | | | GTA[3′]IABkFQ | | CoV-2. 2020. |
| | | | (SEQ ID NO: 3) | | (Herein |
| | | nCoV_IP4-14059Fw | GGTAACTGGTATGATTTCG | 107 bp | incorporated |
| | | | (SEQ ID NO: 4) | | entirely by |
| | | nCoV_IP4-14- | CTGGTCAAGGTTAATA | | reference). |
| | | 14146Rv | TAGG | | |
| | | | (SEQ ID NO: 5) | | |
| | | nCoV_IP4- | [5′]Hex | | |
| | | 14084Probe(+) | TCATACAAA[Zen]CCAC | | |
| | | | GCCAGG | | |
| | | | [3′]IABkFQ | | |
| | | | (SEQ ID NO: 6) | | |
| CPQ_ | CrAssph | 056F1 | CAGAAGTACAAACTCC | 126 bp | 11-Stachler, |
| 056 | age | | TAAAAAA | | E., et al. |
| | | | CGTAGAG | | Quantitative |
| | | | (SEQ ID NO: 7) | | CrAssphage |
| | | 056R1 | GATGACCAATAAACAA | | PCR Assays for |
| | | | GCCATTAGC | | Human Fecal |
| | | | (SEQ ID NO: 8) | | Pollution |
| | | 056P1 | [FAM] | | Measurement. |
| | | | AATAACGATTTACGTG | | Environmental |
| | | | ATGTAAC | | science and |
| | | | [MGB] | | technology |
| | | | (SEQ ID NO: 9) | | 2017, 51, |
| | | | | | (16), 9146- |
| | | | | | 9154). (Herein |
| | | | | | incorporated |
| | | | | | entirely by |
| | | | | | reference). |

Reactions consisted of 12.5 μL TaqMan® Environmental MasterMix (ThermoFisher), 1 μM primers, 80 nM probe, molecular grade water, and 2 μL template DNA for a total reaction volume of 25 μL. Each reaction was run on either a QuantStudio3™ QuantStudio5™ (ThermoFisher) under the following thermal cycling conditions: 10 minutes at 95° C., followed by 40 cycles of 95° C. for 15 seconds and 60° C. for 1 minute. A DNA standard was generated by purification of amplified PCR product using Roche High Pure PCR Template Preparation Kit. Standard DNA quantity was assessed on NanoDrop spectrophotometer and Qubit® fluorometer. A standard curve, including or consisting of purified amplicons ranging from 1×10⁶ to 5 copies/reaction, was used to convert Ct values to gene copies per reaction (see Table 5 below). A CPQ_056 assay modified for SYBR Green chemistry was used in some optimization trials.

TABLE 5

| Assay | R² | Intercept | Slope | Efficiency |
|---|---|---|---|---|
| CPQ_056 | 0.974 | 39.568 | −3.406 | 0.97 |
| IP2IP4 | 0.849 | 41.026 | −3.508 | 0.93 |

### SARS-CoV-2 RT-QPCR

To detect SARS-CoV-2, RT-qPCR was used with a multiplex reaction containing the previously published IP2 and IP4 assays (Table 4). Reactions consisted of 6.25 μL Reliance One-Step Multiplex RT-qPCR Supermix, 0.4 μM each primer, 0.16 μM probes, molecular grade water, and 2.5 μL template total nucleic acids for a total reaction volume of 25 μL. Thermal cycling conditions were 10 minutes at 50° C., 10 minutes at 95° C., followed by 45 cycles of 95° C. for 10 seconds and 59° C. for 30 seconds. A standard curve, consisting of amplicons ranging from 250 to 2.5 copies/reaction, was used to convert Ct values to gene copies per reaction (Table 5). All no-template controls for both crAssphage and IP21P4 (n>40) were negative throughout the entire study.

### Recovery of CrAssphage and SARS-CoV-2

To assess the proportion of viral nucleic acids remaining in each phase (including the pellet) after centrifugation, wastewater samples (2×20 ml) were spiked with SARS-CoV-2 (resulting in approximately 580 gene copies per ml wastewater). Pellets were generated by ultracentrifugation at 150,000×g for 45 minutes and the following layers were removed; aqueous upper (top 10 ml), aqueous lower (second 9 ml), cushion interface (1.5 ml, specifically targeting visible suspended particles on top of sucrose layer), sucrose upper (6 ml), sucrose lower (6 ml), and pellet (appx. 200 μL). A 200 μL aliquot of each layer was extracted using the PowerViral kit (Qiagen) and extracts were analyzed with the IP2IP4 assay in 25 μL reaction volumes under the conditions described previously.

### Cumulative Incidence of COVID-19

To correlate evidence of SARS-CoV-2 transmission in wastewater with cases of COVID-19 in the wastewater catchment area we retrieved the total number of COVID-19 cases by ZIP code from the Upstate Hospital electronic medical record system. These records reflect approximately 40% of the total COVID-19 cases in Onondaga County. The number of COVID-19 cases was standardized by a geographic location , in this case by ZIP code to the ZIP code population to calculate a cumulative incidence per 1,000 residents. The cumulative incidence was visualized alongside the ratio of log₁₀(SARS-CoV-2): log₁₀(crAssphage).

### Results

### Recovery of Viral Nucleic Acids

Direct ultracentrifugation of a wastewater sample through a % 50 sucrose cushion resulted in the formation of a translucent, but visible, pellet often bordered by darker, lower density residue, presumably organics, metal sulfides, and/or other impurities in the wastewater (See e.g., FIG. 4A and 4B).

Depending on the wastewater sample, lower density impurities were often resting at the cushion interface. Recovery trials with inactive SARS-CoV-2 spiked samples indicated that no quantifiable SARS-CoV-2 RNA, crAssphage RNA, or crAssphage DNA remained in the upper aqueous phase or sucrose layer after centrifugation (See e.g., Table 6).

TABLE 6

| | | | SARS-CoV-2 |
|---|---|---|---|
| | Replicate | Mean Copies SARS-CoV-2 | Rxns Positive |
| Layer | Tube | (+/−SD) | (out of three) |
| Aqueous | 1 | <LOQ | 0 |
| Upper | 2 | <LOQ | 0 |
| Aqueous | 1 | <LOQ | 0 |
| Lower | 2 | <LOQ | 0 |
| Cushion | 1 | <LOQ | 0 |
| Interface | 2 | <LOQ | 2 |
| Sucrose | 1 | <LOQ | 0 |
| Upper | 2 | <LOQ | 0 |
| Sucrose | 1 | <LOQ | 0 |
| Lower | 2 | <LOQ | 0 |
| Pellet | 1 | 1.37E3 (7.39E2) per pellet | 3 |
| | 2 | 1.42E3 (7.19E2) per pellet | 3 |

Given that only a portion of each layer was tested, there may have been some non-pelleted residual viral nucleic acids at concentrations below the limit of detection, but it is clear that the vast majority of SARS-CoV-2 and crAssphage viral nucleic acids are pelleted under these conditions. After nucleic acid extraction and qPCR, recovery of SARS-CoV-2 is estimated based on the addition of a known quantity of SARS-CoV-2 to be about 12% attributing some loss to non-pelleted viral RNA, but most to the subsequent nucleic acid extraction procedure.

### Yield and Quality Assessment of Total RNA

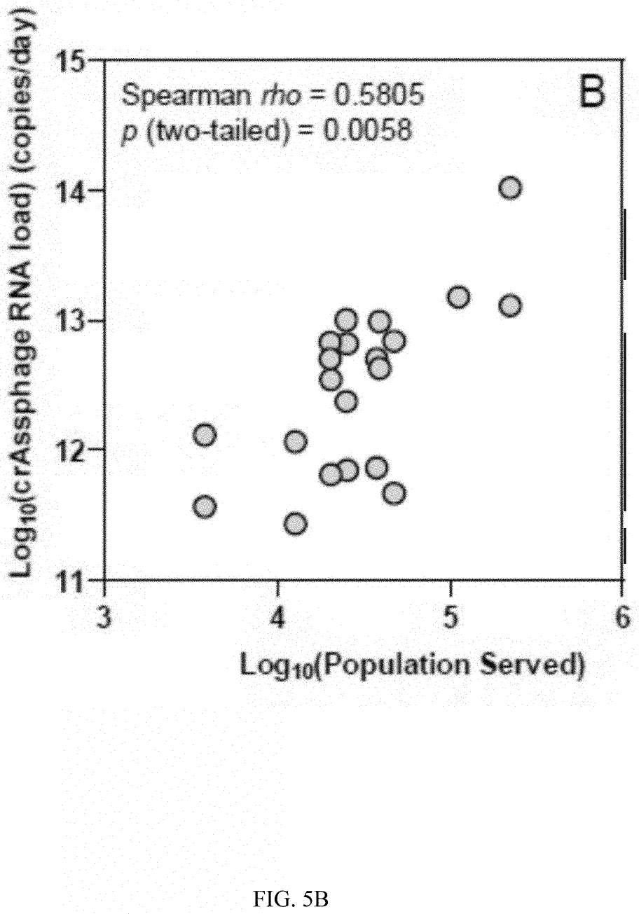

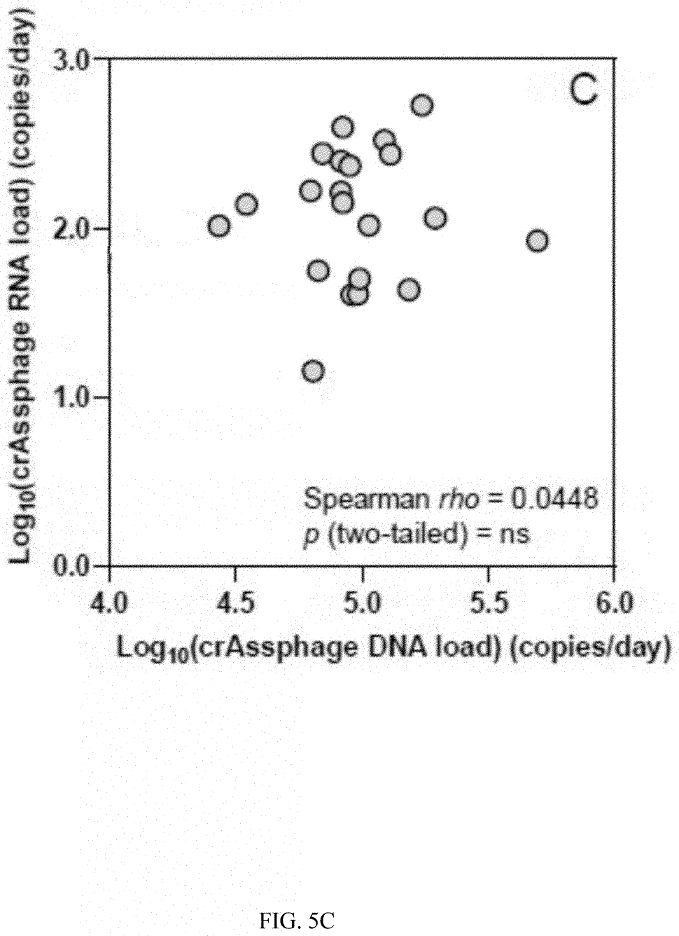

The direct ultracentrifugation and purification of wastewater resulted in recovery of a considerable amount of total RNA. In the samples that were collected on May 6 the average yield was 26.3 ng/μL (std. dev. =11.7) (FIG. 5, top panel). However, these estimates were likely affected by the presence of considerable DNA carryover. Thus, for the May 13 samples, the addition of DNase produced lower yields, with an average of 2.5 ng/μL (std. dev.=2.3) (FIG. 5, bottom panel). The RNA integrity numbers of the samples were not significantly different in either batch, however, with May 6 average of 4.3 ng/μL (std. dev.=0.8) and a May 13 average of 3.4 ng/μL (std. dev.=1.9) (FIG. 5). Notably, these RNA integrity values are similar to ones obtained from human biofluid waste products, such as saliva, urine, or fecal matter. Accordingly, Table 6 demonstrates recovery of SARS-CoV-2 RNA through a sucrose cushion.

### Detection and Quantification of Viral Nucleic Acids in Wastewater

Over a two-week period, some level of SARS-CoV-2 were detected in 18 out of 22 samples, 13 of which were in the quantifiable range (See e.g., Table 7).

TABLE 7
Concentrations of SARS-CoV-2 and crAssphage DNA and RNA in sampled catchments.

| | | | | | SARS-CoV-2 | |
|---|---|---|---|---|---|---|
| | | Mean copies crAssphage | | | Mean Copies | |
| Date | Facility/ | (CPQ_056)/mL wastewater (+/−SD) | | | SARS-CoV-2/mL | Rxns Positive |
| Collected | Catchment | DNA | RNA | | (+/−SD) | (cut of three) |
| May 6 | 600 | 5.79E4 (3.58E3) | 2.76E2 | (4.50E0) | <LOQ | 0 |
| May 6 | 601 | 1.76E5 (3.36E4) | 5.33E2 | (1.33E1) | 1.12E2 (8.01E0) | 3 |
| May 6 | 603 | 5.44E4 (4.03E3) | 1.65E2 | (3.75E1) | 1.51E1 (7.89E0) | 3 |
| May 6 | 604 | 9.29E4 (5.65E3) | 3.95E2 | (5.08E1) | 4.59E1 (1.29E1) | 3 |
| May 6 | 605 | 1.19E5 (1.26E4) | 3.31E2 | (7.55E1) | <LOQ | 0 |
| May 6 | 606 | 7.36E4 (1.47E3) | 2.48E2 | (4.61E1) | <LOQ | 2 |
| May 6 | 610 | 3.33E4 (1.19E3) | 1.37E2 | (1.59E1) | 2.25E1 (1.45E1) | 3 |
| May 6 | 617 | 1.47E5 (8.78E3) | 2.73E2 | (5.12E1) | <LOQ | 2 |
| May 6 | 619 | 7.58E4 (1.18E4) | 1.62E2 | (2.35E1) | <LOQ | 0 |
| May 6 | 725 | 1.01E5 (8.28E3) | 2.32E2 | (2.99E1) | <LOQ | 0 |
| May 6 | 1700 | 2.50E4 (2.25E3) | 1.02E2 | (2.30E1) | <LOQ | 1 |
| May 13 | 600 | 1.07E5 (6.97E3) | 1.03E2 | (2.44E1) | 7.50E0(2.94E0) | 3 |
| May 13 | 601 | 8.44E4 (7.8E33) | 1.40E2 | (3.44E1) | 2.82E1 (4.67E0) | 3 |
| May 13 | 603 | 6.43E4 (1.43E3) | 1.32E1 | (6.52E0) | 3.04E1 (3.14E1) | 3 |
| May 13 | 604 | 6.74E4 (1.03E3) | 5.50E1 | (1.57E1) | 5.72E1 (2.62E1) | 3 |
| May 13 | 605 | 9.15E4 (1.81E3) | 3.94E1 | (1.57E1) | 1.20E1 (2.39E0) | 3 |
| May 13 | 606 | 9.65E4 (1.38E3) | 4.00E1 | (1.60E1) | 4.90E1 (6.33E0) | 3 |
| May 13 | 610 | 2.21E4 (1.02E3) | <LOQ | (0/3) | 6.92E1 (7.89E0) | 3 |
| May 13 | 617 | 9.81E4 (3.15E3) | 4.00E1 | (1.17E1) | <LOQ | 1 |
| May 13 | 619 | 1.54E5 (7.35E3) | 4.21E1 | (8.88E0) | <LOQ | 1 |
| May 13 | 725 | 1.95E5 (7.73E3) | 1.13E2 | (7.16E0) | 1.30E1 (8.34E0) | 3 |
| May 13 | 1700 | 4.96E5 (3.66E3) | 8.29E1 | (3.43E1 | 9.41E1 (2.00E1) | 3 |

SARS-CoV-2 was more prevalent in the May 13 samples (detected in 11 out of 11 samples) compared to the May 6 samples (7 out of 11), possibly due to a significantly lower flow on May 13 (paired t-test, p=0.045). If any of the three reaction wells crossed the fluorescence threshold, the sample was interpreted as positive due to all negative controls throughout the study testing negative. Likewise, SARS-CoV-2 fell within the quantifiable range in 9 out of 11 May 13 samples and only 4 out of 11 May 6 samples. The average number of SARS-CoV-2 genome copies within quantifiable samples over the two-week period was 42.7 (std.dev=32.9) genomes/ml while the highest observed was 112.35 (std. dev.=8.01) genome/ml of wastewater.

In contrast, crAssphage DNA was abundant in every sample analyzed with an average of 1.11×10⁵ copies/ml across the two-week period with no significant difference between the two sample sets. crAssphage RNA was detected in every sample except the sample from Facility 610 on May 13. crAssphage RNA was much less abundant than crAssphage DNA with an average of 1.68×10² copies/ml. Interestingly, while there was no significant difference in crAssphage DNA between the two sample sets, crAssphage RNA was significantly lower on May 13 than May 6 (paired and unpaired t-tests, p<0.001).

Association Between crAssphage Loads and Population Served

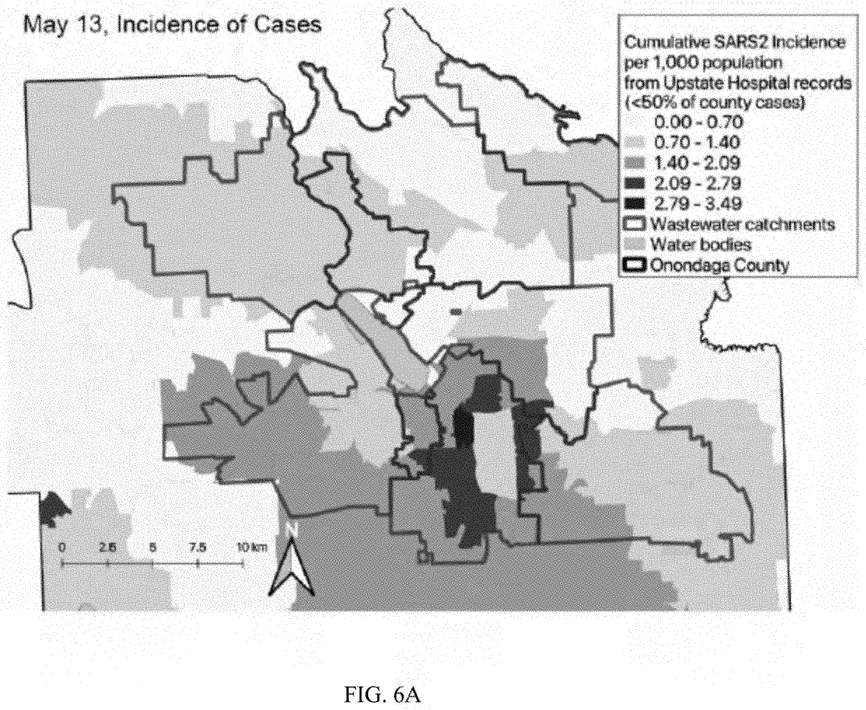

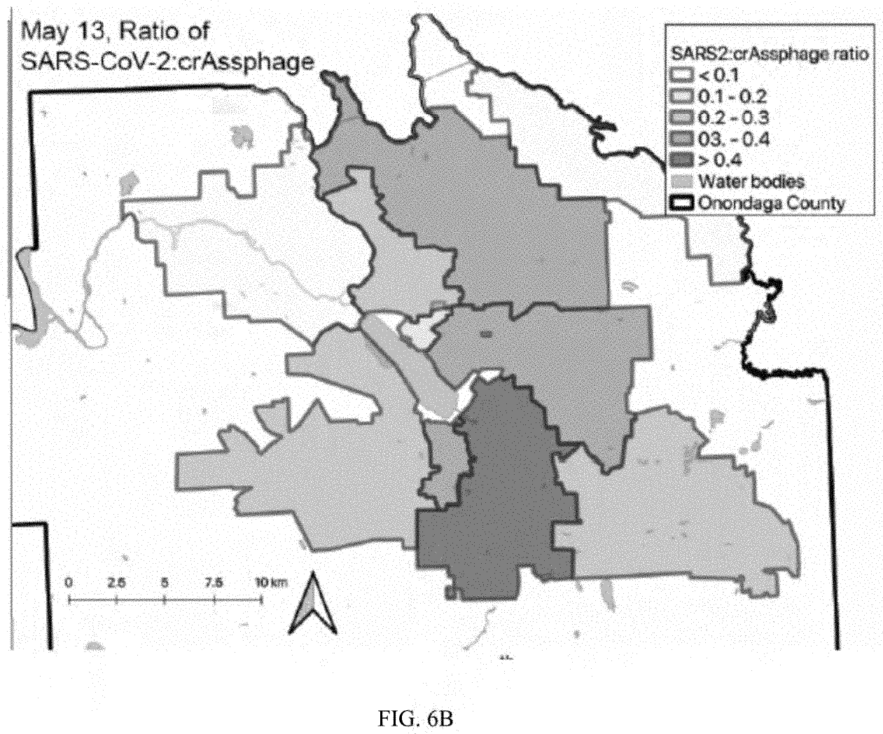

While crAssphage DNA concentrations were not significantly associated with population served, flow, SARS-CoV-2 concentrations, or crAssphage RNA concentrations, there was a significant linear relationship between the loads of both crAssphage DNA (p<0.001) and RNA (p<0.01) and population served in each catchment (FIGS. 6A-6C).

### Spatial Association Between SARS-CoV-2:CrAssphage Ratios in Wastewater and CO VID-19 Incidence

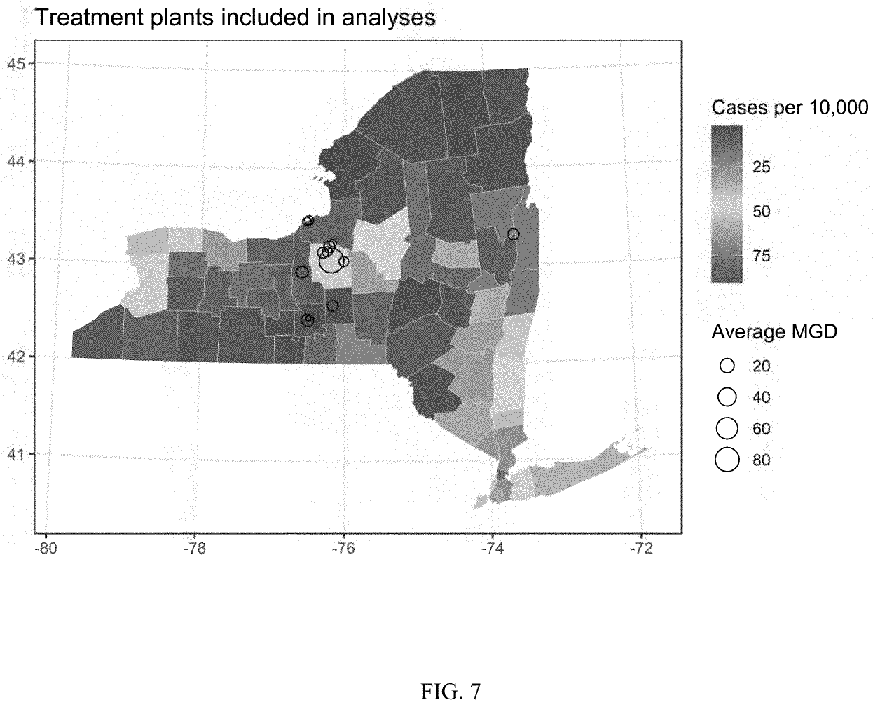

Although the number of cases in each catchment would allow a better assessment of the relationship between viral concentrations in wastewater and the level of transmission in the respective community, visual inspection suggests a spatial correlation between the cumulative incidence of cases by zip code from the Upstate hospital system and the ratio of SARS-CoV-2:crAssphage in wastewater with higher ratios occurring in areas or geographical locations of higher incidence FIGS. 7A and 7B).

### Discussion

The first demonstration that surveillance of SARS-CoV-2 in wastewater could be used to inform the public health response to COVID-19 instantly expanded the tools available to fight the pandemic. However, many recent reports of SARS-CoV-2 detection, or variants thereof, in wastewater are limited by the low levels of viral RNA recovered which can limit quantitative interpretation. Here, a sugar cushion ultracentrifugation method is reported for the recovery of SARS-CoV-2 from wastewater that provided quantitative results from a relatively small sample size (20 ml) in about 8 hours depending on the number of samples processed at one time. It is understood that the requirement of an ultracentrifuge is unrealistic for many and that other approaches will be required in the absence of this equipment. Nonetheless, using a single centrifuge, a modest throughput of about 60 samples in 24 hours with these methods is estimated.

Much of the prior work on crAssphage, specifically those targeted by the CPQ_056 assay, has been focused on its utility as an indicator of human fecal pollution in natural waterbodies. Many of the same attributes that make crAssphage an attractive indicator organism, such as its prevalence and abundance in the human population as well as its scarcity in other hosts, are also helpful in gauging the degree of SARS-CoV-2 transmission within the community. In addition to using crAssphage as an abundant surrogate for SARS-CoV-2 during method development and optimization, crAssphage can be used to ensure sufficient viral recovery, which may become an important quality assurance measure when comparing wastewater surveillance data within and between labs. Furthermore, like SARS-CoV-2, crAssphage is subject to decay and dilution within the wastewater infrastructure and while concentrations of SARS-CoV-2 alone are difficult to interpret, the ratio of SARS-CoV-2:crAssphage is likely more robust to processes that contribute to the loss of viral nucleic acids during transport. It is conceivable that these ratios could then be used to rank catchment areas by their relative degree of transmission independent of mass-balance calculations.

Enveloped RNA viruses, like SARS-CoV-2, are known to be less resilient under environmental conditions than non-enveloped DNA viruses, like crAssphage. Furthermore, DNA released from lysed viral particles is thought to be more resilient to degradation than RNA. The hypothesized rapid decay of SARS-CoV-2 compared to crAssphage would likely result in the underestimation of SARS-CoV-2 transmission within the community when using this approach. Decay rates of the two viruses and their genetic material within water infrastructure are needed to further refine predictions of transmission within the population using this approach. Decay may also play a larger role in larger service areas with longer average wastewater transit times and may explain why only low levels of SARS-CoV-2 were detected in some areas with known cases.

In summary, it has been demonstrated that an ultracentrifugation method using a sugar cushion such as sucrose can be used for quantitative environmental surveillance of SARS-CoV-2 transmission. It has further been shown herein that the ratio of SARS-CoV-2:RNA:crAssphage DNA found in wastewater may be spatially associated with incidence of the disease and could potentially be used to guide public health and economic intervention strategies. Regional or national surveillance of wastewater, in conjunction with clinical testing, may provide a robust decision-making platform that authorities can use to continue restarting local economies while prioritizing public health. Furthermore, frequent and widespread wastewater surveillance has the potential to indicate when and where a resurgence of SARS-CoV-2 or outbreaks of future pathogens might occur.

### Example II

### Sucrose Cushion Alterations

### Concentration and Centrifugation Time Assessment

To optimize the sugar cushion (such as sucrose cushion) purification method, recovery of crAssphage markers was assessed with varying sucrose concentrations and ultracentrifugation times. Three sucrose concentrations were used between 20-70%, namely 20%, 50%, and 70% (in TNE buffer [20 mM Tris-HCl (pH 7.0), 100 mM NaCl, 2 mM EDTA]) with two total replicates of each treatment. Prior to cushion purification, wastewater samples were centrifuged at 2,000×for 25 mins to remove large particles and debris (sample clarification). Six 20 ml aliquots were then distributed to 38.5 ml ultracentrifuge tubes. To the bottom of each tube, 12 ml of a solution of sucrose in TNE was slowly added with a 10 ml serological pipette so that the sucrose formed a distinct layer below the wastewater. A pellet was generated by ultracentrifugation at 4° C. at 150,000×g for between 20 and 150 minutes depending on the sucrose concentration (See Table 2 above). Supernatant was then decanted, and pellets were resuspended in 200 μL 1× PBS (20% treatment) or RNA/ater (50% and 70% treatments). Resuspended pellets were stored at −20° C. for <12 hours prior to nucleic acid extraction using the methods previously described.

A 50% sucrose cushion combined with a 90 minute ultracentrifugation spin time resulted in the highest concentration of recovered crAssphage markers and was thus used for subsequent optimization and wastewater sample processing until it was clear shorter centrifuge times resulted in approximately the same recovery of viral nucleic acids.

### 50% Sucrose Concentration Centrifuge Time Assessment

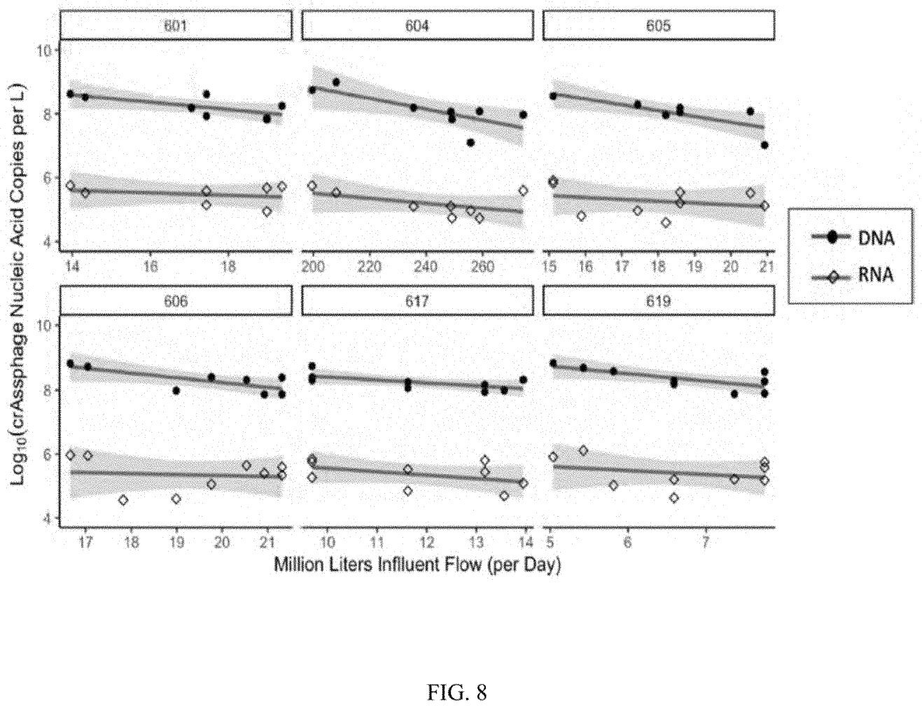

Further optimization was performed by assessing varying spin times with a 50% sucrose cushion (See Table 3 above). Six samples were prepared using the methods previously described, with the omission of the initial 2,000×g clarification spin. Pellets were generated by ultracentrifugation at 4° C. at 150,000×g for 30, 45, and 75 minutes (2 replicates per centrifuge time). Pellets were examined for physical differences (FIG. 8) under the assumption that darker pellets indicated a greater concentration of impurities in the sample. Pellets were resuspended in 200 μL PBS (1×) and stored at −20° C. for <12 hours prior to nucleic acid extraction and cDNA generation using the methods previously described.

A 45-minute centrifugation time resulted in the highest concentration of recovered crAssphage cDNA markers and approximately the same amount of crAssphage DNA markers as longer spins. Due to these recovery data, and the benefit of increased processing throughput provided by a shorter spin time, wastewater samples were purified with 45 minutes of ultracentrifugation starting on May 13th.

Quantification of CrAssphage using SYBR Green Chemistry

For optimization of our sample processing approach, SYBR Green qPCR was used to assess recovery of crAssphage markers. Reactions consisted of 12.5 μL TaqMan Environmental MasterMix, 1 μM CPQ_056 primers, 0.25 μL 10× SYBR Green dye, molecular grade water, and 2 μL template DNA for a total reaction volume of 25 μL. Thermal cycling conditions were 10 minute at 95° C., followed by 40 cycles of 95° C. for 15 seconds and 60° C. for 1 minute followed by a melt curve. A standard curve, consisting of purified amplicons ranging from 1×10⁶ to 5 copies/reaction, was used to convert Ct values to gene copies per reaction (See Table 8).

TABLE 8

| | Detection | | | | |
|---|---|---|---|---|---|
| Assay | Chemistry | R² | Intercept | Slope | Efficiency |
| CPQ_056 | SYBR Green | 0.997 | 35.626 | −3.343 | 0.99 |

### Example III

Introduction—The materials and methods also include those described in Wilder, et al., Co-quantification of crAssphage increases confidence in wastewater-based epidemiology for SARS-CoV-2 in low prevalence, Water Research X 11 (2021) 100100 (herein incorporated by reference in its entirety). It is noted that information in this Example may include information from Example I above, however, additions are provided.

The presence of a substantial quantity of viral RNA in feces and urine provides the opportunity for wastewater-based epidemiology (WBE) approaches to be applied to the surveillance of COVID-19, tracking the emergence of disease, and transmission trends over time. A robust and effective wastewater monitoring program for SARS-CoV-2 is provided that may help to inform resource allocation decisions (e.g. where to prioritize testing and contact tracing), target community interventions such as social distancing measures or other restrictions, and provide an additional tool by which policy makers could assess when and how to reopen local economies. In embodiments, an effective wastewater monitoring approach is provided suitable for use in the surveillance of facilities such as jails, university dormitories, and assisted living facilities which may be especially susceptible to COVID-19 outbreaks. Information obtained from the methods of the present disclosure beneficially provide officials with the tools to limit the spread of the virus both within and from these types of facilities.

To date, large scale monitoring efforts are problematically limited by factors such as turnaround time (e.g. the time from sample acquisition to data generation) and dependence on supply chain continuity for single use materials such as charged membranes or centrifugal filtration units. The inventors have found ultracentrifugation is an attractive approach because once the initial investment in equipment is made, the material cost and sample processing time are low. Furthermore, the ability to manipulate the amount and viscosity of the sedimentation medium as well as the ultracentrifugation time and speed permit partial nucleic acid purification concurrent with concentration.

In embodiments, the present disclosure provides a reliable and scalable method for detecting and quantifying SARS-CoV-2 wastewater RNA (and variants thereof) from areas with low infection rates and/or b) integrate co-quantification of viral nucleic acids from crAssphage, an abundant human gut bacteriophage, into SARS-CoV-2 wastewater monitoring to help account for sources of both inter- and intra-sewershed variability. The inventors have measured both crAssphage DNA and RNA because it is currently unclear which serves as a better fecal normalizer when trying to associate SARS-CoV-2 wastewater RNA concentrations to relevant epidemiological parameters. In embodiments, ultracentrifugation optimization trials were initially conducted prior to analyzing 181 wastewater influent samples collected from six Upstate New York counties.

Materials and methods

### Sampling Locations and Sample Collection and Transport

Twenty-four-hour composite influent wastewater samples (110 mL-1.9 L) were collected from 28 different access points in combined sewage networks across Upstate New York in Onondaga, Cayuga, Cortland, Tompkins, Oswego, and Warren Counties (Table 9, FIG. 8). FIG. 8 depicts a map of wastewater treatment plants included in the analysis reflecting incidence of COVID-19 in New York State Counties and flow rate (millions of gallons per day) of specific facilities.

TABLE 9
Summary of locations and dates of sampling in New York State

| | | Average | Test | N Access | | |
|---|---|---|---|---|---|---|
| | Appx. | Daily | Positivity^{b} | Points | | Total N |
| County | Population | Incidence^{a} | (%) | Sampled | Time Period Sampled | Samples |
| Onondaga | 460,000 | 34.5 | 2.94 | 16 | 4/28-Jun. 24, 2020 | 122 |
| Cayuga | 75,000 | 1.1 | 0.47 | 4 | 5/19-Jun. 22, 2020 | 24 |
| Warren | 70,000 | 0.4 | 0.20 | 3 | 5/27-Jun. 23, 2020 | 15 |
| Oswego | 120,000 | 3.6 | 1.10 | 2 | 6/03-Jun. 23, 2020 | 8 |
| Tompkins | 100,000 | 0.5 | 0.16 | 2 | 6/02-Jun. 22, 2020 | 7 |
| Cortland | 50,000 | 0.2 | 0.11 | 1 | 5/27-Jun. 22, 2020 | 5 |

^{a}Average daily incidence is calculated as the total number of new positive cases that occurred over the time period sampled divided by the length of the time period (days).
^{b}Test positivity is calculated as the total number of positive tests out of the total number of tests performed in each county during the time period sampled. Diagnostic test results include the results of both PCR and antigen tests.

Information on the age of wastewater in these systems was only available for six Onondaga County access points (Table 10), where mean transit time ranged from 1.2 to 4.4 hours. Samples were stored at approximately 4° C. following collection and were transported on ice to Upstate Medical University (Syracuse, N.Y.) the following morning for processing and viral concentration (with the exception of Onondaga County samples collected on the 28th of April, which were frozen at −20° C. for processing at a later date following methodological optimizations). From April 28 to June 24 , 2020 a total of 181 wastewater samples were collected and processed for the detection of SARS-CoV-2 and crAssphage nucleic acids. During the sample collection process, influent flow rate, pH, and water temperature were also measured at some access points. Average daily minimum air temperature in each county during this time period ranged from 9.2 to 14.6° C., average daily maximum air temperature ranged from 21.8 to 25.9° C., average daily precipitation ranged from 0.07 to 0.21 cm, and daily relative humidity ranged from 36 to 86% (NOAA). Information on the topographical area of each sewershed was accessed through New York State and/or County databases and the size of the population served was estimated using census data. Characteristics of individual access points within each county are summarized in Table 10 below.

TABLE 10

| | | N | Population | Area | Transit |
|---|---|---|---|---|---|
| | County | Samples | Served (N | Served | Time |
| Site ID | (NY) | Collected | Individuals) | (Km²) | (Hrs)^{a} |
| CCWW1 | Cayuga | 5 | 31,814 | 42.71 | NA |
| CCWW2 | Cayuga | 5 | 7,374 | 12.16 | NA |
| CCWW3 | Cayuga | 5 | 7,575 | 16.41 | NA |
| CCWW4 | Cayuga | 5 | 17,250 | 12.79 | NA |
| Cortland | Cortland | 4 | 26,950 | 147.29 | NA |
| 600 | Onondaga | 8 | 3,838 | 4.17 | NA |
| 601 | Onondaga | 9 | 25,965 | 29.55 | 1.2 ± 0.4 |
| 603 | Onondaga | 8 | 49,097 | 69.62 | NA |
| 604 | Onondaga | 9 | 242,377 | 253.69 | 2.5 ± 1.3 |
| 605 | Onondaga | 9 | 39,352 | 93.07 | 2.5 ± 1.3 |
| 606 | Onondaga | 9 | 50,727 | 113.6 | 3.5 ± 1.3 |
| 610 | Onondaga | 8 | 128,021 | 73.63 | NA |
| 617 | Onondaga | 9 | 30,613 | 91.56 | 1.8 ± 0.9 |
| 619 | Onondaga | 9 | 16,168 | 58.9 | 4.4 ± 2.3 |
| 725 | Onondaga | 8 | 45,538 | 96.79 | NA |
| 727 | Onondaga | 4 | NA | NA | NA |
| 1700 | Onondaga | 8 | 16,260 | 9.48 | NA |
| 999A | Onondaga | 6 | 87,743 | 60.51 | NA |
| 999B | Onondaga | 6 | 9,577 | 4.31 | NA |
| 999C | Onondaga | 6 | 19,586 | 5.44 | NA |
| 999D | Onondaga | 6 | 11,521 | 3.33 | NA |
| Oswego_E | Oswego | 3 | 12,357 | 68.64 | NA |
| Oswego_W | Oswego | 3 | 14,182 | 14.21 | NA |
| Ithaca | Tompkins | 3 | 60,575 | 100.4 | NA |
| Cayuga | Tompkins | 2 | 13,801 | 147.29 | NA |
| GFI | Warren | 4 | 23,462 | 34.68 | NA |
| SGF | Warren | 4 | 5,603 | 21.07 | NA |
| MBPS | Warren | 4 | 3,572 | 3.41 | NA |

^{a}Transit times calculated by Wang et al 2020.

Ultracentrifugation of Wastewater through a Sucrose Cushion

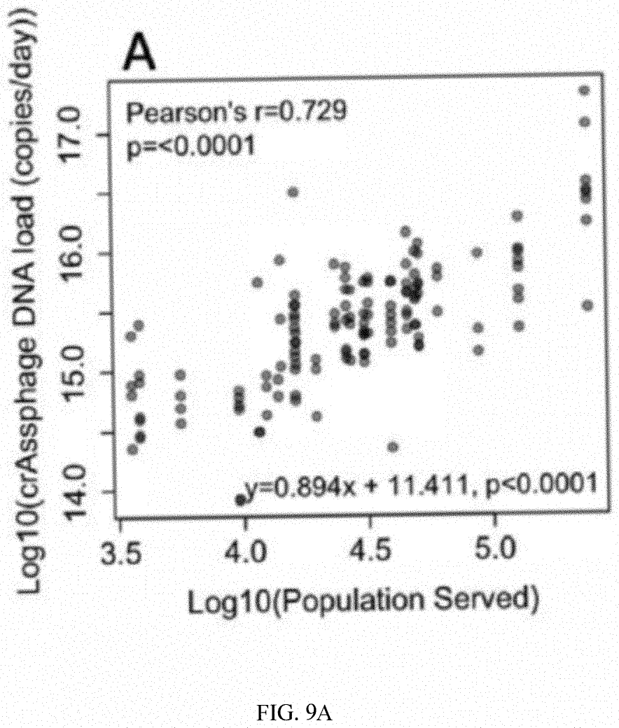

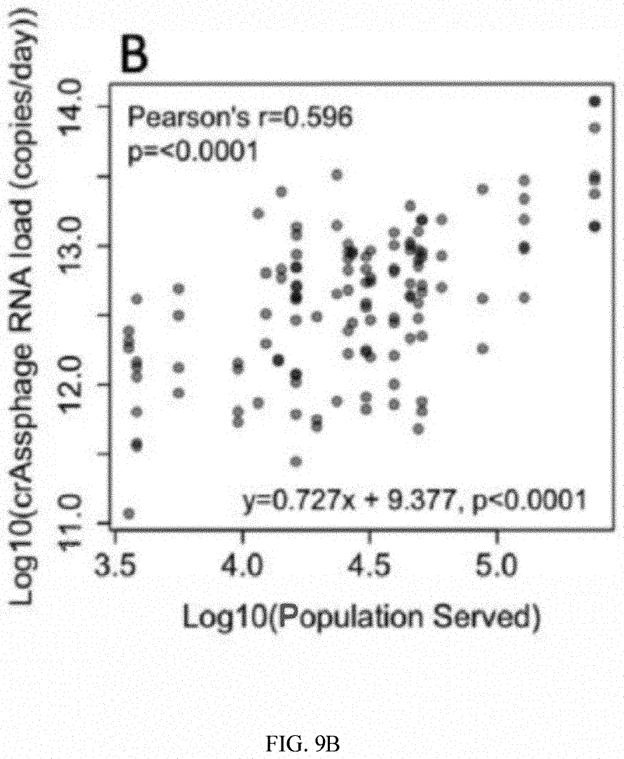

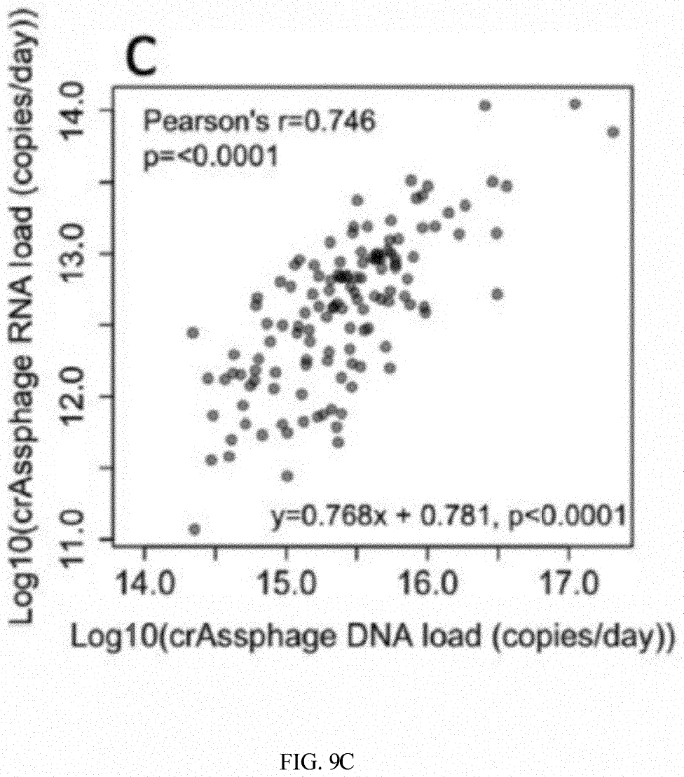

Prior to ultracentrifugation, wastewater samples were blended to resuspend particulates that had settled during transport or storage. Twenty milliliters were transferred into a disposable 38.5 mL ultracentrifuge tube (Product No. 75000471, ThermoFisher®, Mass., USA) using a disposable serological pipette. Unless otherwise noted in the optimization experiments described in the section 2.3, a 12 mL sucrose cushion (50% sucrose in THE buffer [20 mM Tris-HCL (pH 7.0), 100 mM NaCl, 2 mM EDTA]) was then carefully added underneath the wastewater using a serological pipette so that wastewater and the sucrose solution formed distinct layers in the ultracentrifuge tube (FIG. 9A). FIG. 9A depicts raw influent wastewater above disposed atop 50% sucrose solution prior to ultracentrifugation. FIG. 9B depicts a pellet produced by 45 minutes of ultracentrifugation and residual debris on top of the sucrose cushion.

In embodiments, a process sequence of sucrose cushion methodology includes: 1) Sterilize workplace with hydrogen peroxide; 2) thoroughly mix/blend wastewater samples to resuspect particles which may have settled during transport; 3) aliquot 20 mL of wastewater into a 38.5 mL ultracentrifuge tube using a disposable serological pipette; 4) with a new disposable serological pipette, carefully add 12 ml of 50% sucrose solution (or any sucrose solution in accordance with the present disclosure) to the bottom of the ultracentrifuge tube so that wastewater and sucrose are in distinct layers. Repeat for remaining samples; 5) prepare at least one processing blank by substituting distilled or molecular grade water for wastewater (creating a tube with a layer of DI or MG water above sucrose cushion); 6) ensure all tubes are the same mass by adding small quantities of distilled water to the upper layer; 7) ultracentrifuge at 150,000×g and 4° C. for 45 min.; 8) remove supernatant using a pipette. Be careful not to disturb or remove pellet; 9) resuspend pellet in 200 microliter PBS (1×) and transfer to 1.5-2.0 mL microcentrifuge tube; 10) proceed immediately to DNA/RNA extraction or store resuspended pellet at −20 deg. C for extraction within 24 hours; and 11) sterilize workplace and equipment with hydrogen peroxide.

In batches of six, samples were balanced by the addition of distilled water (<500 μL) and then ultracentrifuged at 150,000×g at 4° C. on a Sorvall® WX Ultra series with a Sorvall® SureSpin® 630 (6×36 mL) Swinging-Bucket Rotor (ThermoFisher®). Prepared samples were ultracentrifuged for 45 minutes unless otherwise noted for optimization experiments. Following ultracentrifugation and the generation of pellets containing viral particles and nucleic acids (FIG. 9B), the supernatant was carefully decanted with a new serological pipette and pellets were resuspended in 200 μL 1× PBS and transferred to 1.7 mL microcentrifuge tubes. Resuspended pellets were stored at −20° C. for <24 hours until nucleic acid extraction. Replicates were processed for optimization experiments only.

### Optimization of Viral Nucleic Acid Recovery

Samples collected from Onondaga County on April 28, May 6, and May 13, 2020 were used to perform optimization experiments. To identify sucrose concentrations and ultracentrifugation times that resulted in higher levels of viral nucleic acid recovery, crAssphage was used as a surrogate since native SARS-CoV-2 concentrations were too low to be used as reliable indicator of recovery. First, well-blended wastewater subsamples were ultracentrifuged with 20, 50, and 70% sucrose cushions for 20, 90, and 120 minutes, respectively, with lower concentration cushions receiving shorter ultracentrifugation times. Pellets were then analyzed for crAssphage DNA.

Next, once the optimal sucrose concentration was identified, the effect of reducing ultracentrifugation time was tested by making six replicate subsamples and ultracentrifuging for 30 (n=2), 45 (n=2), and 75 minutes (n=2) while holding sucrose concentration constant. Pellets were then analyzed for both crAssphage DNA and RNA.

Then, to estimate the efficiency with which SARS-CoV-2 RNA is pelleted, 20 mL wastewater aliquots (n =2) with low initial concentrations of SARS-CoV-2 RNA (appx. 8 genome copies per mL) were spiked (spike equivalent to appx. 580 genome copies per mL wastewater) with heat deactivated SARS-CoV-2 (Catalog No. NR-52286, BEI Resources®, Virginia, USA) prior to ultracentrifugation for 45 minutes. After ultracentrifugation, the following layers were analyzed: aqueous upper (top 10 mL), aqueous lower (second 9 mL), cushion interface (1.5 mL, targeting particles suspended just above the sucrose cushion), sucrose upper (6 mL), sucrose lower (6 mL), and pellet (appx. 200 uL). Two hundred microliter subsamples of each layer and the resuspended pellets were then analyzed for recovery of SARS-CoV-2 RNA, crAssphage DNA, and crAssphage RNA.

Finally, to estimate the loss of nucleic acids through the nucleic acid purification procedure specifically, wastewater pellets (n=7) generated from homogenized samples originating from several access points were spiked with appx. 125,000 genome copies of bovine coronavirus (BCoV) RNA extracted from a vaccine (Bovine Rotavirus-Coronavirus Vaccine from Zoetis, N.J., USA). Extraction of total nucleic acids from wastewater pellets was carried out by the method described below. BCoV RNA concentrations were determined via RT-qPCR using a previously published assay See e.g., Decaro, N., Elia, G., Campolo, M., Desario, C., Mari, V., Radogna, A., Colaianni, M. L., Cirone, F., Tempesta, M., Buonavoglia, C., 2008. Detection of bovine coronavirus using a TaqMan-based real-time RT-PCR assay. Journal of Virological Methods 151, 167-171. doi:10.1016/j.jviromet.2008.05.016 (herein incorporated by reference) and Table 11.

TABLE 11
BCoV qPCR assay performance.

| | | | | | LOQ |
|---|---|---|---|---|---|
| Assay | R² | Intercept | Slope | Efficiency | (copies/rxn) |
| BCoV | 0.99 | 37.858 | −3.366 | 0.98 | 10 |

In routine processing, deactivated SARS-COV-2 and bovine coronavirus vaccine were not used to estimate nucleic acid recoveries. Instead, crAssphage was used to confirm recovery of nucleic acids and as a fecal normalizer for SARS-COV-2.

Nucleic Acid Extraction and Synthesis of CrAssphage cDNA

Total nucleic acids were extracted from resuspended pellets using the AlIPrep® PowerViral® DNA/RNA Kit (Qiagen®, Hilden, Germany) according to manufacturer's protocol with the omission of the optional bead beating step. Nucleic acids were eluted in 50 μL elution buffer, five of which was used immediately to generate total cDNA using the QuantiTect® Reverse Transcription Kit (Qiagen®) according to manufacturer's protocol to allow estimation of crAssphage RNA. Total nucleic acid samples and cDNA were immediately stored at −80 □C until viral quantification via RT-qPCR and qPCR.

### Quantification of Viral Nucleic Acids

RT-qPCR was used to detect the presence of SARS-CoV-2 RNA in undiluted total nucleic acid extracts using a multiplex reaction with the previously published IP2 and IP4 assays targeting separate regions of the RdRp gene (Institut Pasteur, 2020). Reactions consisted of 6.25 μL Reliance One-Step Multiplex RT-qPCR Supermix (Bio-Rad®, California, USA), 0.4 μM each primer, 160 nM each probe (both HEX), molecular grade water, and 2.5 μL nucleic acid template for a total reaction volume of 25 μL. Thermal cycling conditions were 10 minutes at 50 deg. C. 10 minutes at 95 deg. C., followed by 45 cycles of 95 deg. C. for 10 seconds and 59 deg. C. for 30 seconds. crAssphage nucleic acids were quantified using the previously published CPQ_056 assay. Reactions consisted of 12.5 μL TaqMan® Environmental MasterMix (ThermoFisher®), 1 μM primers, 80 nM probe, molecular grade water, and 2 μL nucleic acid template for a total reaction volume of 25 μL. Thermal cycling conditions were 10 minutes at 95 deg. C., followed by 40 cycles of 95 deg. C. for 15 second and 60 deg. C. for 1 minute. A standard curve, ranging from 1×10⁶ to 5 copies per reaction of a diluted gBlock® (IDT®, Iowa, USA) containing targets for the IP2IP4 assay or diluted purified crAssphage amplicons (produced with DNA Clean and Concentrator™ 25, ZYMO, USA) was used to convert Ct values to gene copies per reaction. Nucleic acid concentrations for gBlocks and purified amplicons were measured using a Qubit® 3.0 Fluorometer) (Invitrogen®), allowing copy number to be determined from the known length of the amplicon or gBlock. Samples were quantified using either PCR plate specific standard curves or a composite standard curve from recent plates (Table 2). All qPCR reactions were carried out on either QuantStudio® 3 or QuantStudio® 5 (ThermoFisher®) real-time PCR systems.

### Quality Assurance

For all days on which wastewater samples were purified via ultracentrifugation, at least one processing blank was prepared by processing 20 mL distilled water instead of wastewater and measuring levels of SARS-CoV-2 RNA, crAssphage DNA, and crAssphage RNA. Throughout the study, 2 of 22 processing blanks contained quantifiable levels of crAssphage DNA (mean Ct=36.019±2.259) which was several orders of magnitude less than crAssphage quantities obtained from wastewater influent samples (mean Ct=22.662±1.418). For SARS-CoV-2, 2 of 22 processing blanks showed some degree of amplification, with one being quantifiable (Jun. 10, 2020, Ct=35.637±0.192, appx. 10 copies/mL). No processing blanks had amplification for crAssphage cDNA.

Following ultracentrifugation, at least one additional blank was prepared during total nucleic acid extraction by substituting 200 μL dissolved pellet for 200 μL molecular grade water. Throughout the study, 1 of 18 extraction blanks contained quantifiable levels of crAssphage DNA (Ct=35.982±0.545). For SARS-CoV-2, 1 of 18 extraction blanks contained quantifiable RNA (Jun. 9, 2020, Ct=34.323±0.515, appx. 24 copies/mL). No extraction blanks contained detectable levels of crAssphage cDNA. Due to suspected SARS-CoV-2 contamination, data from 9 and 10 of Jun. 2020 were omitted from our analysis, effectively reducing the number of samples analyzed from 181 to 169.

For RT-qPCR and qPCR, plates contained at least three no template control (NTC) reactions. Throughout the study, 1 of 128 (0.8%) IP2IP4 NTC wells amplified (Ct=40.957) and 9 of 198 (4.5%) of CPQ_056 NTC wells amplified. For CPQ_056, six of these NTC amplifications occurred on May 11, 2020. On this plate, wastewater samples had a mean CPQ_056 Ct of 23.350 and positive NTCs had a mean Ct of 38.580 which suggests that contamination did not greatly affect estimates of crAssphage from wastewater on this run. Therefore, since the sole IP2IP4 NTC that showed amplification was >40 cycles and the few positive CPQ_056 NTCs were largely isolated to one plate and represented DNA quantities several orders of magnitude less than our wastewater nucleic acid extracts, no data were excluded from analysis based on the assessment of NTCs.

Kinetic outlier detection (KOD) was performed as described previously (Green and Field, 2012; Kirtane et al., 2019; Tichopad et al., 2010) on all 3,032 reactions to determine if qPCR inhibition affected the amplification of SARS-CoV-2 or crAssphage nucleic acid targets. Raw fluorescence data from each well were log-transformed and fit to a 4-parameter sigmoidal model using the perbatch function in R package qpcR version 1.4-1 (Ritz and Spiess, 2008; Spiess, 2018). The first and second derivative maxima of each fitted model was then estimated. Using a 10 Ct difference between the first and second derivative maxima as a quality criterion (i.e., “uni2” criteria in function perbatch), KOD analysis indicated that all wells with a Ct value <45 (maximum possible Ct value) displayed no signs of inhibition. Inherent in these methods is the assumption that DNA polymerase and reverse transcriptase are equally susceptible to PCR inhibitors. Nonetheless, the total absence of signs of qPCR inhibition as indicated by sensitive KOD methods suggests that the purification methods used were effective at removing compounds that commonly affect qPCR amplification.

### Integration of COVID-19 Case Data

COVID-19 testing data, including all diagnostic tests including PCR and antigen-based methods, were obtained from The Electronic Clinical Laboratory Reporting System (ECLRS) (NYSDOH, 2020a,b). All test results were classified as positive, negative, inconclusive, or invalid. After excluding tests for out of state residents, all tests for COVID-19 during the study period were geocoded using the New York State Street Address Maintenance (SAM) Program (“NYS Street Address Mapping (SAM),” 2020). Additional geocoding was performed with geocoders from SAS and MapMarker to improve accuracy. Shape files of each service area were obtained from each corresponding municipality. Addresses occurring within the studied service areas were retained while addresses occurring outside the study area were excluded. Residences with private septics were identified using statewide tax parcel data from the New York State GIS Clearinghouse (“NYS GIS-Parcels,” 2020). Any addresses with private septics, which accounted for approximately 5% of COVID-19 tests, were excluded from the analysis. A daily count of positive test results by service area was tabulated after excluding inconclusive or invalid results. Human subject involvement with regards to COVID-19 diagnostic testing was approved by the New York State Department of Health's Institutional Review Board.

### Data Interpretation and Analysis

To aid interpretation, SARS-CoV-2 wastewater RNA levels were classified into three distinct categories prior to data analysis. Samples that had all three qPCR replicates amplify above the LOQ of 5 genome copies per reaction were classified as quantifiable. Because both assays were able to amplify 5 copies per reaction consistently, samples that had at least one qPCR replicate amplify with a Ct <40 were considered detected but not quantifiable (DNQ). Many of the samples classified as DNQ had one or two qPCR replicates above the LOQ of 5 copies but were still conservatively classified as DNQ for our analysis. Samples that had no amplification in any of the three wells (i.e., all three wells were “Undetermined”, Ct >45) were considered below the limits of detection (BLOD; i.e., a “negative” sample).

To facilitate comparison of crAssphage concentrations between service areas, we used prior 24-hour flow and population serviced to calculate a per capita crAssphage nucleic acid load as follows:

$$Per Capita Nucleic Acid Load = \frac{Genome Copies per Liter \times Daily Flow ( L )}{Population Served ( n persons )}$$

Per capita nucleic acid load represents the estimated average daily contribution of crAssphage nucleic acids by an individual.

Pair-wise t-tests were used to test for significant differences in mean recovery using different sucrose concentrations and spin times. Conditional inference trees (CTrees) were developed using the partykit (version 1.2-10) package in R (version 1.2.5019) as done previously (Weller et al., 2020) to assess the effects of service area size, average influent temperature, and pH on crAssphage DNA and RNA concentrations (R code available one the world wide web at github.com/Maxwell-Wilder/Co-quantification-of-crAssphage-increases-confidence-in-wastewater-based-epidemiology-for-SARS-CoV-2). Transit times were also used a predictor variable, but only for sites 601, 604, 605, 606, 617, and 619 as available (Wang et al., 2020).

### Results

### Optimizing Recovery of Viral Nucleic Acids

In an assessment of sucrose concentration and ultracentrifugation (“spin”) time, it was found that a 50% cushion paired with a 90-minute spin time yielded greater crAssphage DNA concentrations than both a 20% cushion paired with a 20-minute spin time and a 70% cushion paired with a 150-minute spin time (p<0.05, See Table 12 below).

TABLE 12

| Sucrose | Spin Time | Replicate | crAssphage DNA (Copies/ |
|---|---|---|---|
| Concentration | (Minutes) | Tube | L WW Source +/− SD) |
| 20% | 20 | 1 | 1.95 × 10⁷ (4.40 × 10⁵) |
| | | 2 | 2.53 × 10⁷ (5.95 × 10⁵) |
| 50% | 90 | 1 | 9.04 × 10⁷ (3.56 × 10⁶) |
| | | 2 | 1.27 × 10⁸ (1.88 × 10⁶) |
| 70% | 150 | 1 | 3.26 × 10⁷ (3.41 × 10⁵) |
| | | ² | 7.52 × 10⁷ (8.55 × 10⁵) |

The impact of 30, 45, and 75-minute spin times was then assessed on crAssphage nucleic acid recovery using a 50% sucrose cushion in an attempt to reduce processing time. It was found that while both 45 and 75-minute spin times yielded greater quantities of crAssphage DNA than a 30-minute spin (p=<0.01, See Table 13), there was no significant difference in crAssphage DNA recovery between 45 and 75-minute spins. Quantifiable amounts of crAssphage RNA were only recovered with a 45-minute spin time (Table 13).

TABLE 13

| | | crAssphage DNA | crAssphage RNA |
|---|---|---|---|
| Spin Time | Replicate | (Copies/L WW | (Copies/L WW |
| Minutes) | Tube | Source +/− SD) | Source +/− SD) |
| 30 | 1 | 6.74 × 10⁷ (1.57 × 10⁶) | DNQ |
| | 2 | 7.21 × 10⁷ (4.47 × 10⁶) | BLOD |
| 45 | 1 | 9.89 × 10⁷ (4.16 × 10⁶) | 3.46 × 10⁴ |
| | | | (6.72 × 10³) |
| | 2 | 9.90 × 10⁷ (2.82 × 10⁶) | DNQ |
| 75* | 1 | 9.39 × 10⁷ (5.86 × 10⁶) | DNQ |
| | 2 | 1.19 × 10⁸ (3.80 × 10⁶) | DNQ |

*Among-treatment crAssphage DNA recovery was statistically different only for the 75-minute spin time (p = 0.005).

Low quantities of crAssphage RNA (DNQ) are potentially due to degradation, as RNA may have degraded while the wastewater sample was stored at 4° C. for approximately 5 days. Because 75-minute and 45-minute spin times yielded similar results, with 45-minutes being the only treatment to recover quantifiable crAssphage RNA, we proceeded with this spin time for further experiments and the analysis of wastewater samples.

Having determined an optimal sucrose concentration and ultracentrifugation time, the approximate nucleic acid recovery was determined for the total process using spiked heat deactivated SARS-CoV-2 (BEI Resources® and native crAssphage DNA and RNA as surrogates. Quantifiable levels of SARS-CoV-2 RNA and crAssphage RNA were found only in the pellet indicating that the majority of viral RNA is likely pelleted under these conditions (See Table 14 below).

TABLE 14

| | Volume | Replicate | SARS-CoV-2 RNA | crAssphage RNA | crAssphage DNA |
|---|---|---|---|---|---|
| Layer | (mL) | Tube | (Copies +/− SD) | (Copies +/− SD) | (Copies +/− SD) |
| Aqueous | 10 | 1 | BLOD | BLOD | DNQ |
| Upper | | 2 | BLDD | BLOD | DNQ |
| Aqueous | 9 | 1 | BLOD | BLOD | 2.30 × 10² (6.36 × 10¹) |
| Lower | | 2 | BLOD | BLOD | DNQ |
| Cushion | 15 | 1 | BLOD | BLOD | 4.10 × 10³ (7.07 × 10¹) |
| Interface | | 2 | DNQ | BLOD | 8.59 × 10³ (3.97 × 10²) |
| Sucrose | 6 | 1 | BLOD | DNQ | 1.13 × 10⁴ (3.92 × 10²) |
| Upper | | 2 | BLOD | BLOD | 4.72 × 10³ (5.43 × 10³) |
| Sucrose | 6 | 1 | BLOD | DNQ | 1.88 × 10⁴ (1.24 × 10³) |
| Lower | | 2 | BLOD | DNQ | 8.19 × 10³ (4.71 × 10²) |
| Pellet | 0.2 | 1 | 1.37 × 10³ (7.39 × 10²) | 1.60 × 10³ (3.57 × 10²) | 2.26 × 10⁶ (7.93 × 10⁴) |
| | | 2 | 1.42 × 10³ (7.19 × 10²) | DNQ | 3.54 × 10⁶ (1.80 × 10⁵) |

Trace levels of RNA recovered from the cushion interface (SARS-CoV-2) and in the sucrose layers (crAssphage) suggest that low levels of SARS-COV-2 RNA also remain unpelleted. While quantifiable levels of crAssphage DNA were present in most layers post-ultracentrifugation, quantities found in the pellet were far greater than that of any other layer (p<0.001, See table 14). Although the magnitude of crAssphage DNA recovered from the pellet varied significantly among the two replicates (p=0.002), SARS-CoV-2 RNA recovery was not statistically different. Based on the amount initially spiked, we estimated that 12% (s.d.=5.5%) of deactivated SARS-CoV-2 RNA was recovered after both ultracentrifugation and nucleic acid extraction processes. A follow-up experiment in which BCoV RNA was added to pellets resulted in an average extraction recovery of 6.89% (s.d.=1.58%) suggesting that the majority of nucleic acid loss in the total process occurred at the nucleic acid extraction step.

### Abundance of SARS-CoV-2 and CrAssphage in Wastewater Samples

While the vast majority of crAssphage DNA and RNA values fell within the quantifiable range, most SARS-CoV-2 RNA levels were either DNQ (49%) or BLOD (34%, Table 15).

TABLE 15

| Target | Quantifiable (%) | DNQ (%) | BLOD (%) |
|---|---|---|---|
| crAssphage DNA | 100 | 0 | 0 |
| crAssphage RNA | 93 | 6 | 1 |
| SARS-CoV-2 RNA | 17 | 49 | 34 |

Detecting or quantifying SARS-CoV-2 RNA from wastewater was obtained in 111 of the 169 samples that were analyzed over the study period. Of these 111 samples, 29 had quantifiable levels of SARS-CoV-2 RNA. In these samples, the average quantity of SARS-CoV-2 RNA recovered was 2.16×10⁴ (s.d.=2.11×10⁴) genome copies per L of wastewater while the maximum observed quantity was 1.02×10⁵ (s.d.=7.96×10³) genome copies per L of wastewater.

crAssphage nucleic acids were quantifiable from the vast majority of wastewater samples (Table 15). Over the course of the study period, the average and maximum quantities of crAssphage DNA recovered were 2.05×10⁸ (s.d.=2.18×10⁸) and 1.73×10⁹ (s.d.=7.72×10⁷) genome copies per L of wastewater. For crAssphage RNA, the average and maximum quantities recovered were 4.00×10⁵ (s.d.=4.61×10⁵) and 2.88×10⁷ (s.d.=1.47×10⁵) genome copies per L.

Association between CrAssphage Loads, Influent Flow, and Population Served

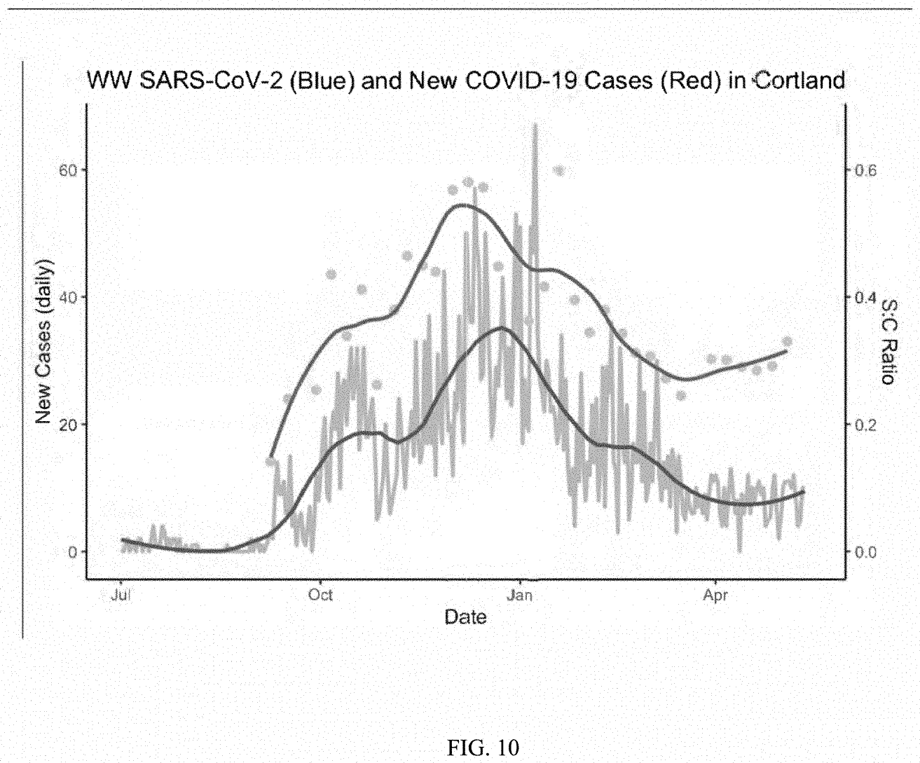

A significant negative relationship was observed between crAssphage concentrations detected and influent wastewater flow rates across all sites (Table 16). Table 16 depicts regression parameters for the relationship between crAssphage nucleic acid (log₁₀ copier per L wastewater) concentration and flow (millions of liters per day) at six sites (corresponds to FIG. 10).

TABLE 16

| | crAssphage | Regression | | p |
|---|---|---|---|---|
| Site | Nucleic Acid | Equation | R² | value |
| All | DNA | Y = −0.0013X + | 0.027 | 0.031 |
| | | 8.197 | | |
| | RNA | Y = −0.0018X + | 0.046 | 0.007 |
| | | 5.442 | | |
| 601 | DNA | Y = −0.115X + | 0.418 | 0.050 |
| | | 10.201 | | |
| | RNA | Y = −0.039X + | −0.109 | 0.549 |
| | | 6.146 | | |
| 604 | DNA | Y = −0.017X + | 0.512 | 0.028 |
| | | 12.254 | | |
| | RNA | Y = −0.008X + | 0.142 | 0.192 |
| | | 7.085 | | |
| 605 | DNA | Y = −0.183X + | 0.606 | 0.014 |
| | | 11.384 | | |
| | RNA | Y = −0.057X + | −0.060 | 0.484 |
| | | 6.291 | | |
| 606 | DNA | Y = −0.149X + | 0.481 | 0.034 |
| | | 11.223 | | |
| | RNA | Y = −0.031X + | −0.130 | 0.784 |
| | | 5.935 | | |
| 617 | DNA | Y = −0.092X + | 0.360 | 0.052 |
| | | 9.333 | | |
| | RNA | Y = −0.106X + | 0.084 | 0.229 |
| | | 6.611 | | |
| 619 | DNA | Y = −0.233X + | 0.450 | 0.029 |
| | | 9.913 | | |
| | RNA | Y = −0.125X + | −0.054 | 0.467 |
| | | 6.230 | | |

Six sites were selected with the greatest number of sampling events (n=9 each) to look at this relationship on an individual basis and found significant negative relationships between crAssphage DNA concentration and flow, but no significant relationship between crAssphage RNA concentration and flow, potentially due to increased variability in crAssphage RNA measurements (FIG. 10, Table 16). Lower crAssphage concentrations during higher flow rates are likely attributable to wastewater dilution though sources such as groundwater infiltration and stormwater runoff, although the relative contribution of these sources in each service area is difficult to quantify. Significant relationships between crAssphage DNA and RNA loads and the population served in each service area were found.

Variability in Physical-Chemical Parameters and CrAssphage Loads between Service Areas

At each site (n=8) where temperature and flow data were recorded, a significant positive correlation was observed between temperature and sampling date (Pearson's r=0.94-0.97, p<0.05) and significant negative correlation between flow and sampling date (r=−0.82-−0.96, p<0.05) reflecting the change toward warmer, dryer weather. At facilities 605 and 606, we also observed a significant negative correlation between pH and both sampling date (r=−0.75 , −0.86, p<0.05) and water temperature (r=−0.76 , −0.88, p<0.05), but at other sites no significant correlation was observed.

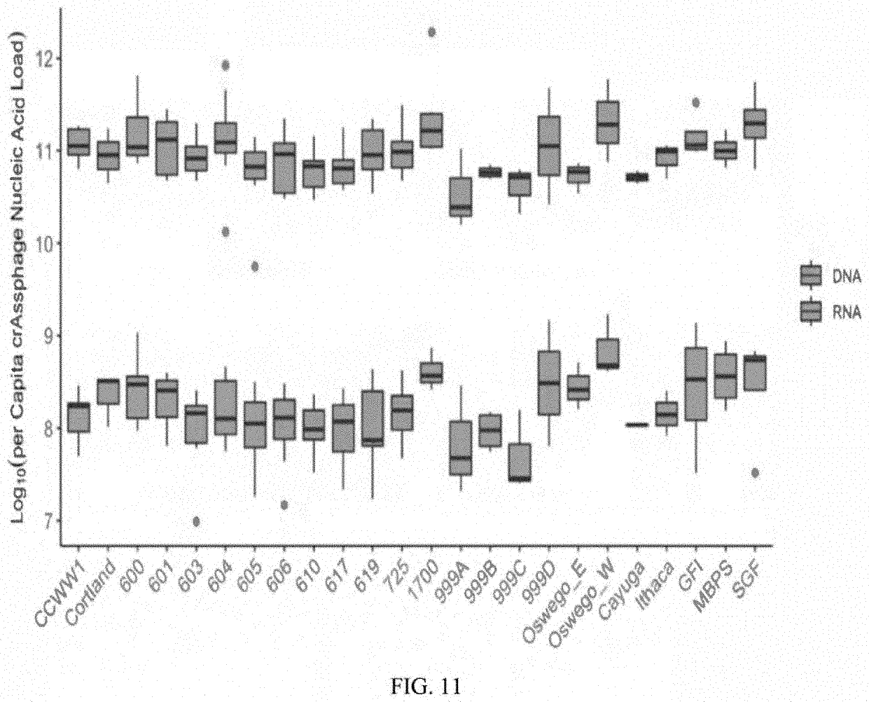

On average, estimated per capita crAssphage contributions were 1.35×10¹¹ genome copies per day (std. dev.=1.99×10¹¹) and 2.42×10⁸ genome copies per day (std. dev.=2.77×10⁸) for DNA and RNA, respectively (FIG. 11). Based on regression analysis, we observed significantly lower per capita crAssphage DNA in service area 999A (4.85×10¹⁰, p=0.0365) compared to other sites. Significantly higher crAssphage RNA per capita in service area Oswego_W (2.86×10¹¹, p=0.0165) was observed compared to other sites. Based on a pairwise comparison of service areas, no two sites had a significant difference in mean crAssphage DNA (p>0.11; Tukey HSD, 95% CI) or mean crAssphage RNA (p>0.09), although the sample size (Table 10) is too low at most sites to conclude no difference in means.

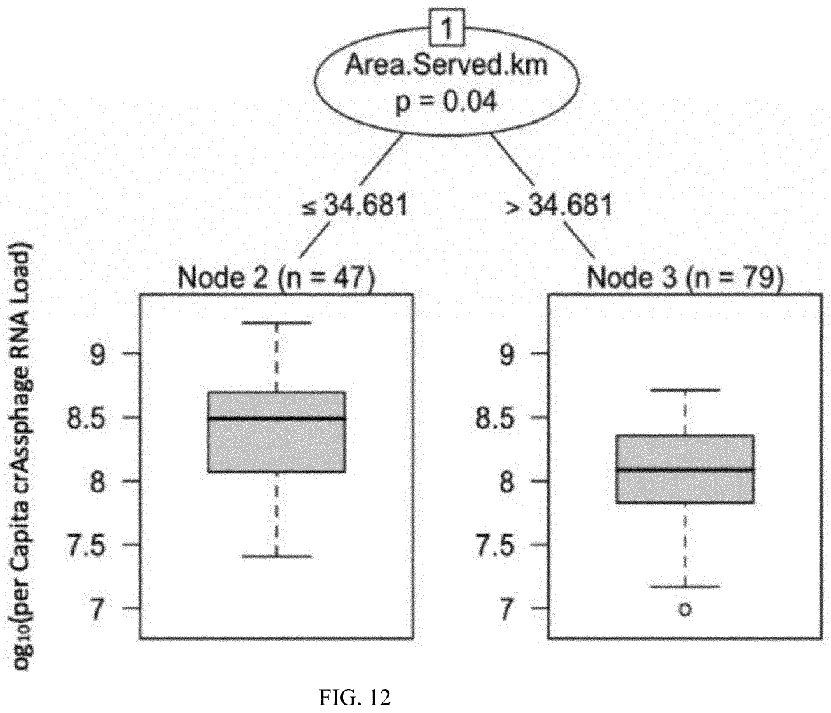

Using CTrees, variability in per capita crAssphage RNA loads could be explained by service area size. Per capita crAssphage RNA loads were 2.09×10⁸ gene copies higher in service areas smaller than 34.681 km² (FIG. 12). A similar association was not observed for per capita DNA loads. No significant splits were identified when using average influent temperature, pH, or transit time as predictors for DNA or RNA loads as outcomes.

Association between SARS-CoV-2 Concentrations and COVID-19 Incidence Following Wastewater Sample Collection

Overall study areas, the highest positive test rates for which SARS-CoV-2 RNA remained BLOD corresponded to a weekly positive test rate of 12.4% and a weekly average of 2.19 daily positive tests per 10,000 population. The highest weekly positive test rates for samples classified as BLOD or DNQ were 20.9% or 3.97 daily positive tests per 10,000 population. Weekly positive test rates corresponding to quantifiable samples ranged from 1.68-15.11% or 0.37-5.95 daily positive tests per 10,000 population depending on the site.

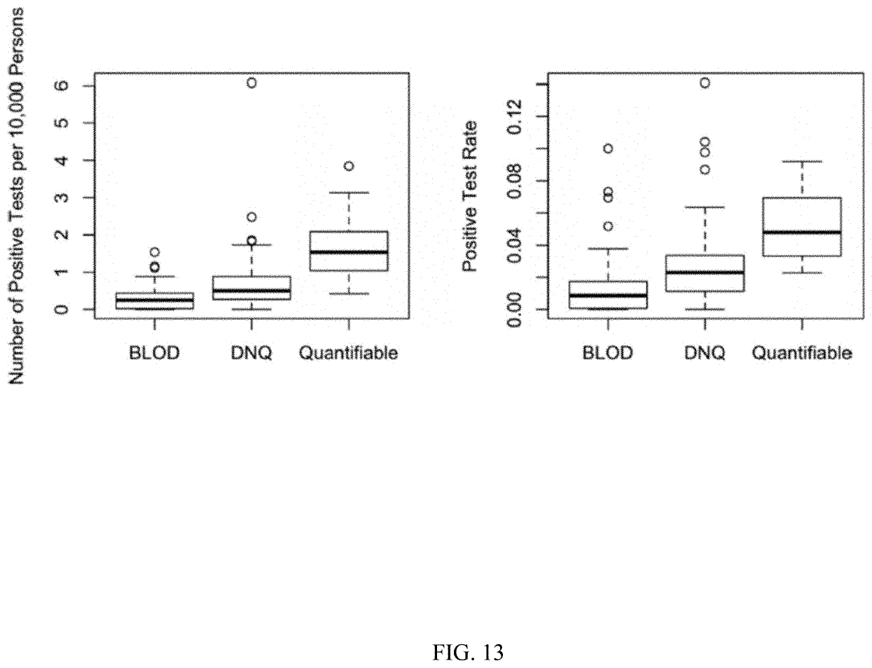

From a qualitative perspective, samples with quantifiable levels of SARS-Cov-2 RNA were associated with higher levels of positive test results the week following sampling (FIG. 13). Over the seven days following wastewater sample collection, both the average number of new positive tests per 10,000 persons and the testing positivity rate were significantly higher in quantifiable samples than in samples classified as BLOD or DNQ for SARS-CoV-2 (Welch two-sample t-test, p<0.001). Samples classified as DNQ also had significantly higher rates and case counts than BLOD samples (Welch two-sample t-test, p≤0.002).

Although the number of samples with quantifiable levels of SARS-CoV-2 RNA was limited (n=29), we did observe a significant relationship between the ratio of SARS-CoV-2 RNA to crAssphage DNA (p=0.005, R²=0.27) and the number of positive tests per 10,000 population. This relationship was somewhat improved after excluding samples for which no crAssphage RNA was recovered (p=0.004, R²=0.31). A similar association was found between the SARS-CoV-2:crAssphage DNA ratio and the number of positive tests per 10,000 population the week following sampling (p=0.003, R²=0.30), which also improved slightly after excluding samples with no recoverable crAssphage RNA (p=0.004, R²=0.33). Interestingly, significant positive associations between ratios and test rates were identified only when testing was expressed as a proportion of the population served and not as a proportion of total tests conducted (i.e., test positivity). No significant linear associations were identified between SARS-CoV-2 wastewater RNA concentrations and epidemiological parameters without first normalizing to crAssphage DNA.

### Discussion

Advantages of Ultracentrifugation through a Sucrose Cushion

A sensitive, rapid, and scalable method has been developed for the detection and quantification of SARS-CoV-2 wastewater RNA based on direct ultracentrifugation though a sugar cushion such as a sucrose cushion. The methods capitalize on the ability of ultracentrifugation to remove low-density contaminants that could potentially interfere with subsequent nucleic acid extraction and qPCR. Despite loss of RNA in the extraction process, SARS-CoV-2 was quantified in areas with less than 1 positive test per 10,000 individuals. With this approach, it is possible to obtain wastewater testing results for both SARS-CoV-2 and crAssphage within 4.5 hours (such as less than 5 hours) of a sample being received. The major limiting factor to this method is both ultracentrifuge availability and capacity, as the rotor used here can hold only six samples. However, the use of small volumes of wastewater (only 20 mL per sample) provides advantages in terms of transport, storage, and biosafety, although the use of larger volumes of wastewater may improve sensitivity. Other groups have since found the method to outperform other common concentration procedures for the analysis of wastewater from individual facilitie. This sensitivity, relatively quick turnaround time, and limited dependence on supply chain continuity may be an attractive option for groups considering wastewater surveillance.

Different concentration and nucleic acid extraction approaches should also be considered in an attempt to improve upon the 7-12% recovery that we estimated in this study. While approaches other than ultracentrifugation have reported higher recovery rates and variable cost and processing times (Table 17), Colosi and colleagues (2020) recently found sucrose cushion-based ultracentrifugation using a fixed-angle rotor, which accommodates tubes with twice the sample volume used in this study, and a NucleoSpin° extraction kit to outperform both electropositive filtration and PEG-precipitation methods. While the ultracentrifugation approach described by Colosi et al. (2020) demonstrated success, it is difficult to compare our methodologies without a direct measurement of nucleic acid percent recovery.

TABLE 17
Comparison of concentration methods for SARS-CoV-2 wastewater surveillance
Table 17

| | | | Estimated | | |
|---|---|---|---|---|---|
| | Appx. | Material | Viral | | |
| | Turnaround | Cost per | Nucleic | Target for | |
| Concentration | Time^{a} | Sample^{b} | Acid | Recovery | |
| Method | (hrs) | (USD) | Recovery | Estimation | Reference |
| PEG | 14-18 | 16.18 | 0.28 ± 0.10% | PMMoV | (Gerrity et al., |
| Precipitation | | | 11 ± 8.4% | and BCoV | 2021) |
| | 6.42 | 22.63 | 44.0 ± 27.7% | MHV | (Ahmed et al., |
| | | | | | 2020b) |
| | 14-18 | 33.01 | 2.04 ± 0.70% | HCoV 229E | (La Rosa et al., |
| | | | | | 2020) |
| Aluminum | 4.33 | 12.00 | 11 ± 2.1% | MgV and | (Randazzo et |
| Flocculation | | | 11 ± 3.5% | PEDV | al., 2020) |
| | 4.58 | 18.05 | 45 ± 19% | FCV | (Barril et al., |
| | | | | | 2021) |
| Centrifugal | 3.67 | 53.19 | 73 ± 53% | F-specific | (Medema et al., |
| Filtration | | | | RNA | 2020) |
| | | | | phages | |
| | 3.58 | 53.19 | 28 ± 9.1% | MHV | (Ahmed et al., |
| | 3.58 | 32.19 | 56 ± 32.3% | MHV | 2020b) |
| | 4.58 | 48.41 | 1.3 ± 0.16% | PMMoV | (Gerrity et al., |
| | | | 55 ± 38% | and BCoV | 2021) |
| High Flow | Not | 31.97 | 4.0 ± 2.2% | PMMoV | (Gerrity et al., |
| Ultrafiltration | Reported | | 54 ± 11% | and BCoV | 2021) |
| | Not | 42.17 | 22 ± 4% | OC43 | (McMinn et al., |
| | Reported | | | | 2021) |
| Electronegative | 3.25 | 18.89 | 26.7 ± 15.3% | MHV | (Ahmed et al., |
| Membranes | | | to | | 2020b) |
| | | | 65.7 ± 23.8% | | |
| Sucrose- | 4.50 | 23.45 | 7-12% | BCoV and | This Study |
| Cushion | | | | SARS-CoV-2 | |
| Ultracentrifugation | | | | | |

^{a}Turnaround time defined as the length of time between sample receipt and RT-qPCR data.
^{b}Processing cost does not reflect the cost of items such as centrifuges, rotors, refrigerator/freezers, qPCR machines, disposables, etc. This estimation also is made assuming all approaches have the same RT-qPCR cost and that reactions are performed in triplicate.

Results from optimization trials indicate that viral particles in wastewater exist in a mixture of states and a range of sedimentation properties that are likely to change from sample to sample. More sensitive methods, but also improved interpretation of wastewater surveillance data, would be facilitated by a better understanding of the state of both SARS-CoV-2 RNA and surrogate nucleic acids within wastewater and, specifically, what proportion are a) contained within viral particles, b) released and dissolved, or c) released and bound to other particles. While some studies have explored viral associations to various wastewater particles in terms of size and charge variability in particle association between different types of viruses suggests that both SARS-CoV-2 and surrogate viral particle associations may require specific study with an additional focus on the state(s) of nucleic acids. If some DNA or RNA is bound, knowing the size and mass of the particles and how they vary over time and across locations would greatly improve the precision of methods based on size (e.g., ultrafiltration), charge (e.g., electropositive filtration), or mass (e.g., ultracentrifugation). Additionally, variability in wastewater particle composition between service areas and/or sampling locations may affect viral decay, as different particle associations have been shown to impact the survival of pathogens (as reviewed in Chahal et al. 2016). Bivins et al., (2020) estimated that 90% of SARS-CoV-2 RNA is degraded after 3.3 days in wastewater. A better understanding of how particle associations alter decay these rates is needed.

### Epidemiologically Relevant Limits of Detection

Although method performance varied across sites, SARS-CoV-2 wastewater RNA could be quantified in some areas experiencing as low as a 1.68% positivity rate. The method's ability to quantify such low levels of SARS-CoV-2 RNA suggests that the results are likely to be useful in managing public health responses at the initial stages of community spread, making this an important public health tool for COVID-19 surveillance. Observation that SARS-CoV-2 RNA detection was associated with a higher incidence of COVID-19 in the next seven days further supports the use of wastewater surveillance as an early warning system with the amount of early warning dependent on a wide range of factors including frequency of wastewater sampling, site and sample characteristics affecting the sensitivity of detection, and the rate of the spread of infection. Further characterization of the service areas themselves and their wastewater infrastructure is needed to determine more precisely the areas where WBE for SARS-CoV-2 would be most useful.

### CrAssphage as a Normalizer for Spatiotemporal Variability

Quantification of a surrogate organism in addition to SARS-CoV-2 can not only serve as a quality assurance measure, ensuring sufficient amounts of nucleic acids are recovered, but can also be used to normalize measured SARS-CoV-2 values to help account for the fluctuating concentrations of fecal material in wastewater. Observation that SARS-CoV-2:crAssphage DNA ratios were significantly associated with the number of positive tests per 10,000 individuals, both 7 days before and after sampling, supports the use of crAssphage as a surrogate for SARS-CoV-2. As the significance of this association was improved following the exclusion of samples from which crAssphage RNA was not recovered, the quantification of both DNA and expressed RNA may be advantageous when using a DNA virus as a surrogate. Other viruses, such as pepper mild motile virus (PMMoV), have been used to facilitate the interpretation of SARS-CoV-2 WBE data. Despite being an RNA virus like SARS-CoV-2, PMMoV is a rod-shaped virus that is very stable in the environment and has high temperature tolerance and resilience in adverse physiochemical conditions (Kitajima et al., 2018), which likely contributes to its relatively consistent abundance across wastewater treatment facilities (D′Aoust et al., 2021b, 2021a). In contrast, results herein show that crAssphage nucleic acid concentrations are somewhat reflective of site differences and that crAssphage DNA concentrations respond to changes in flow and crAssphage RNA fluctuates in part as a function of sewershed area. Lower crAssphage RNA levels from larger sewersheds likely reflects the relative instability of RNA and suggests that measures of sewershed area, or other proxies for waste transit time, could help link SARS-CoV-2 wastewater RNA levels to relevant epidemiological parameters.

The assay used to target crAssphage, CPQ_056, has been shown to cross-react with poultry litter (Ahmed et al., 2018). However, CPQ_056 marker concentrations were over 2-3 orders of magnitude lower in poultry litter compared to untreated wastewater and likely had little effect on our quantification of crAssphage nucleic acids. Nonetheless, use of this marker in areas heavily affected by poultry fecal contamination is not recommended.

### Conclusions

The ultracentrifugation-based method described here is a rapid and sensitive approach for the detection of SARS-CoV-2 in wastewater from areas with low numbers of COVID-19 cases. In embodiments, after normalization with crAssphage DNA, higher concentrations of SARS-CoV-2 wastewater RNA were significantly associated with positive COVID-19 tests the week following wastewater sample collection suggesting the approach could help predict near-term COVID-19 case levels.

### Example IV

### Ultracentrifugation Alterations

### Increased Sensitivity by Increasing Volume of Wastewater Analyzed

Studies were initially conducted using 38.5 ml tubes containing 12 ml sucrose cushion and 20 ml wastewater. Subsequent studies were conducted using the F37L-8x100 rotor equipped with 100 ml tubes that could hold 17 ml of sucrose cushion and 45 ml wastewater (37,000 rpm [182,500×g] for 32 minutes) thereby more than doubling the sensitivity of the method.

### Nucleic Acid Extraction

Studies were conducted using a Qiagen PowerViral/Allprep Kit for nucleic acid extraction. However, the availability of this extraction method may be limited, and alternative methods were investigated for efficient recovery of nucleic acids. The ZYMO Quick-RNA Fungal/Bacterial Microprep Kit resulted in the best recovery of nucleic acids and was used for all subsequent analyses. Briefly, after ultracentrifugation and removal of the supernatant, 800 ul RNA Lysis buffer is added directly to the pellet. The dissolved pellet is then transferred to 2 ml tubes and stored at −20 C (same day extraction) or −80 C (next day extraction) before extraction following the manufacturer's procedure. Nucleic acids are eluted in 30 ul DNase/RNase-free water and used immediately or stored at −80 C.

### Integration of Bovine Coronavirus Spike-and-Recovery System

A spike and recovery system comprised of a commercially available bovine coronavirus vaccine and a previously developed qPCR assay has been incorporated into the workflow to estimate the recovery of nucleic acids. Briefly, lyophilized vaccine (Zoetis, VLN190/PCN 1931.20) containing bovine coronavirus RNA (publicly available NC_003045.1) is mixed with 500 ul filter-sterilized 1× PBS to create single-use aliquots of approximately 10⁶ copies per ul. Ten microliters is added directly to the wastewater sample upon receipt. Following ultracentrifugation and nucleic acid extraction the previously developed bovine coronavirus assay (‘BCoV’, Decaro et al 2008) is used to quantify the amount of recovered spike to provide a percentage recovery.

### Variant Detection

### 69-70 S Deletion Assay

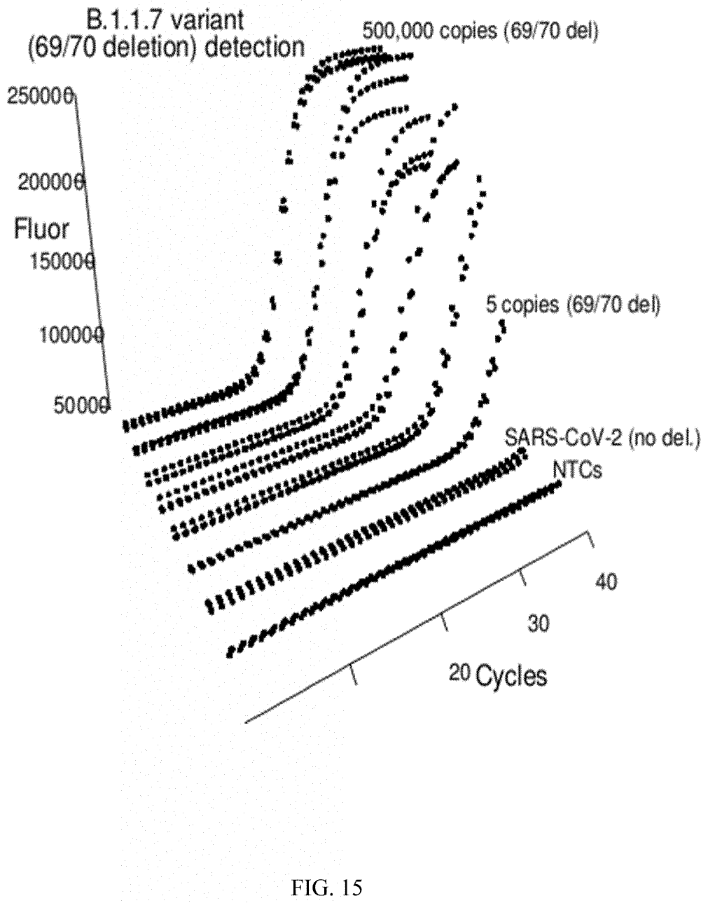

There is a tremendous need to monitor viral variants within the population that may confer increased transmissibility or immune escape. Therefore, a qPCR assay has been developed targeting the 69-70 S deletion that is common in the SARS-CoV-2 variant B.1.1.7 (Alpha' or previously, the ‘UK’ variant) that can be integrated into the wastewater analysis workflow (Table 21, FIG. 14). Other assays that target this mutation are available (See e.g., Chantal Vogels, Joseph Fauver, Nathan Grubaugh 2021. Multiplexed RT-qPCR to screen for SARS-COV-2 B.1.1.7, B.1.351, and P.1 variants of concern. protocols.io located on the world wide web at dx.doi.org/10.17504/protocols.io.br9vm966. However, these commonly use a ‘dropout’ system that is incompatible with samples that may contain a mixture of variants, like wastewater. It is now demonstrated that a) the assay of the present disclosure is both sensitive and specific to variants containing the 69-70 S deletion (Table 22, FIG. 15) and b) the assay can be integrated into the wastewater workflow to estimate the approximate proportion or maximum proportion of infections attributable to variants containing the 69-70 S deletion (Table 23).

### Sequencing

In addition to monitoring for specific individual nucleotide changes, generating whole genome sequence data for wastewater samples is demonstrated, using an optimized sequencing protocol modified from the ARCTICv3 method, that achieves whole-genome viral sequence coverage sufficient to clearly identify variant lineages (FIG. 16), even when the Ct value for detection of the virus exceeds 35 cycles (FIG. 17).

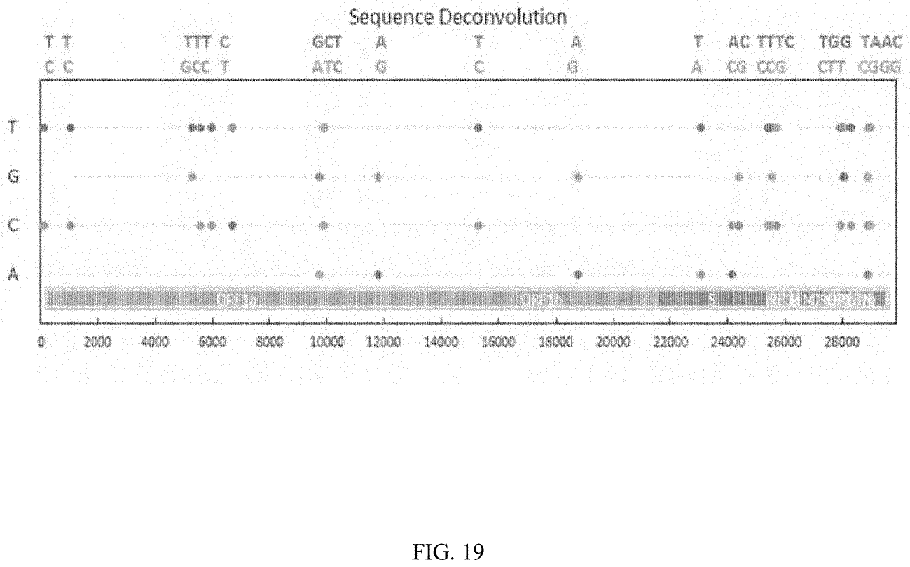

In embodiments, the present disclosure includes a sequencing and variant analysis protocol, including one or more of the steps below:
1. The purity and concentration of each nucleic acid is checked by UV spectrophotometry, and recorded for QC purposes.
2. The composite RNA plate is then subjected to the optimized SARS-CoV-2 sequencing protocol, which incorporates the ARCTICv3 method. Briefly, samples are subjected to reverse transcription in 20 uL volumes using NEB LunaScript MasterMix, followed by multiplex PCR using Q5 HostStart High-Fidelity 2x MasterMix (NEB) with the ARCTIC Primer Pools 1 and 2 (from IDT) in 24 uL volumes. Once complete, plates are evaluated for amplification using a PicoGreen assay to ensure PCR yields in the expected range and diluted by addition of molecular biology grade water to approximately 2.5 ng/uL. These diluted DNA samples are then processed using the plexWell 384 Library Preparation Kit (Seq Well), enabling a single technician to complete library synthesis (including sample barcoding, pool barcoding, library amplification, and purification) of 282 samples plus positive and negative controls in 1.5 days.
3. Sequencing library sizes are assessed using the High Sensitivity DNA Kit on the Agilent Bioanalyzer 2100, quantified with the Qubit fluorometer using the Invitrogen Qubit dsDNA HS Assay Kit, and sequenced on the Illumina MiSeq or NextSeq 500 instrument, using a NextSeq Mid Output Kit (for up to 3 pools) or a NextSeq High Output Kit (for up to 9 pools) with paired end 2x151bp reads containing 1% PhiX spike-in. This requires approximately 25 hours to complete, and typically generates >130M paired-end (Mid-Output) or >400M paired-end (High-Output) reads with more than 80% of all bases exceeding a QC phred score of 30.
4. After sequencing is completed, data are converted to base calls in the SUNYMAC core using the Illumina Bcl2FastQ in a Linux command line format and uploaded to Illumina BaseSpace using the command line interface. In BaseSpace, the Dragen COVID Lineage application (v.3.5.2) is used to perform QC analysis as well as genome assembly, variant calling and annotation, and FASTQ file generation. The results of this are examined for various QC metrics, including high viral kmer counts, lack of host kmer counts, coverage depth, missingness, and mutation numbers, which are downloaded in their entirely for deposition into the sample specific database.
5. FASTQ sequences are uploaded to GISAID NextClade for confirmation of all sequence and variant calls obtained by Dragen, and sequences visualized with the web-based clustering tools to map divergence of the different lineages.
6. For sequence deconvolution, the raw FASTQ sequences are subjected to adapter and index trimming, followed by alignment to the SARS-CoV-2 genome (accession number NC_045512.2) using Bowtie2 or the BWA algorithm. Aligned sequences are then used to call variants using SamTools and FreeBayes open source tools compared to the reference sequence. Only high-quality variants found with both programs are carried through to the next stage.
7. Where variants were observed, the majority of reads are used to identify the primary variant sequence present in the sample (top sequence in blue, FIG. 19) and the minority of reads were used to identify the secondary variant sequence present (bottom sequence in orange, FIG. 19).
8. Variants that are present in at least 1% of the raw reads are then evaluated for specificity in different viral strains. The overall proportion of each variant in the mapped strain-specific (or strain-informative) reads is then estimated by multiplying the percent of alternate allele-specific reads and the percent of total allele-specific reads in that sample out of the total mapped reads.
   An example of the results of these calculations is shown for one representative wastewater sample obtained in Syracuse N.Y., March 2021. This sample yielded an estimate of 1% UK variant reads and 5.8% NY variant reads. The remaining viral variants identified did not help delimit the fractions any further in this specific case.

### Tables

TABLE 21
Primer names and sequences for the 69-70 S deletion assay.

| Primer Name | Sequence (5′-3′) | Reference |
|---|---|---|
| Yale/ESF_69/70_F_01 | CAA TGT TAC TTG GTT CCA TGC TAT | Modified from |
| | CTC (SEQ ID NO: 10) | Vogels et al 2021 |
| ESF_69/70_R_01 | CCA GCC TCT TAT TAT GTT AGA CTT | This study |
| | CTC AG (SEQ ID NO: 11) | |
| ESF_69/70_P_01 | [Cy5] GGT ATC AAA [TAO] CCT CTT AGT | This study |
| | ACC ATT GGT CCC AGA GA [IBRQ]* | |
| | (SEQ ID NO: 12) | |

*IDT oligo entry format: /5Cy5/GGTATCAAA/TAO/CCTCTTAGTACCATTGGTCCCAGAGA/3IAbRQSp/(SEQ ID NO: 13

TABLE 22
Performance parameters of the 69-70 S deletion
assay on synthetic DNA standards.
Table 22

| Assay | | | | | |
|---|---|---|---|---|---|
| Name | R² | Intercept | Slope | Efficiency | LOQ |
| ESF_69/70 | 0.999 | 38.132 | −3.404 | 96.68% | 5 |

TABLE 23
Use of newly developed 69-70 deletion assay to estimate the prevalence or maximum
prevalence of variants containing the 69-70 deletion from wastewater. In cases where the
69-70 deletion was not detected, the limit of detection/quantification (5 copies/reaction)
was used to estimate the maximum prevalence. If the 69-70 deletion was in the quantifiable
range, prevalence of variants containing the 69-70 deletion was estimated based on the
proportion of ESF_69-70: IP2/IP4.
Table 23

| | | SARS-CoV-2 (IP2/IP4, | 69-70 deletion | | Maximum |
|---|---|---|---|---|---|
| | Date Sample | Wuhan strain) | (ESF_69-70) | Prevalence | prevalence |
| Sample ID | Received | copies/mL | copies/mL | (%) | (%) |
| WW-4025 | Feb. 26, 2021 | 117.0 | Not | NA | 0.6 |
| | | | Detected | | |
| WW-4026 | Feb. 26, 2021 | 3.0 | Not | NA | 22.3 |
| | | | Detected | | |
| WW-4058 | Mar. 1, 2021 | 39.5 | Not | NA | 1.7 |
| | | | Detected | | |
| WW-4066 | Mar. 2, 2021 | 18.2 | Not | NA | 3.7 |
| | | | Detected | | |
| WW-4072 | Mar. 2, 2021 | 26.4 | Not | NA | 2.5 |
| | | | Detected | | |
| WW-4073 | Mar. 2, 2021 | 34.1 | 0.895 | 2.6 | NA |

TABLE 24
Sequencing QC and alignment metrics for wastewater and reference samples.

| | | Sample | Virus Load | Total | Total | Aligned |
|---|---|---|---|---|---|---|
| Sample name | Sample Source | PCR date | (copies/uL) | reads | alignments | % |
| P18A11_S177 | Syracuse University | Apr. 4, 2021 | 255.6 | 181145 | 45226 | 13.42 |
| P18A12_S185 | SUNY Oneonta | Mar. 28, 2021 | 25.4 | 113660 | 10025 | 5.05 |
| P18811_S178 | Onondaga County | Mar. 28, 2021 | 11.1 | 46585 | 36171 | 39.51 |
| P18812_S185 | SUNY Oswego | Apr. 4, 2021 | 44.9 | 79152 | 29955 | 19.78 |
| P18C11_S179 | Siena College | Mar. 28, 2021 | 65.7 | 56481 | 9653 | 9.27 |
| P18C12_S187 | St. John Fisher | Mar. 21, 2021 | 72.8 | 22067 | 8338 | 19.91 |
| P18D11_S180 | RIT | Mar. 28, 2021 | 50.6 | 36999 | 30522 | 41.96 |
| P18D12_S188 | Syracuse University | Mar. 14, 2021 | 723.8 | 132706 | 222640 | 84.56 |
| P18E11_S181 | Oswego City | Apr. 4, 2021 | 27.1 | 68679 | 21291 | 16.10 |
| P18E12_S1S9 | RIT | Jan. 31, 2021 | 46.8 | 88986 | 112981 | 64.21 |
| P18F11_S182 | Cortland City | Mar. 28, 2021 | 12.4 | 50578 | 61238 | 61.40 |
| P18F12_S190 | Oneonta City | Jan. 31, 2021 | 79.3 | 106634 | 7123 | 3.81 |
| P18G11_S183 | Oneonta City | Apr. 4, 2021 | 30 | 24193 | 17365 | 36.87 |
| P18H11_S184 | Jefferson County | Apr. 4, 2021 | 45.4 | 32796 | 35690 | 55.31 |
| P18G12_S191 | Wuhan Reference RNA | Jan. 1, 2020 | 50 | 325501 | 631631 | 97.75 |

| | Total | Total | Total | | Avg. | | |
|---|---|---|---|---|---|---|---|
| | unaligned | unique | unique | Coverage | coverage | Avg. | Avg. |
| Sample name | reads | singleton | paired | % | depth | length | quality |
| P18A11_S177 | 156829 | 3406 | 20910 | 99.2 | 194.1 | 136.4 | 32.4 |
| P18A12_S185 | 107924 | 1446 | 4290 | 97.9 | 40.5 | 134.6 | 32.9 |
| P18811_S178 | 28179 | 643 | 17764 | 94.4 | 169.1 | 135.3 | 33.1 |
| P18812_S185 | 63499 | 1351 | 14302 | 96.0 | 133.4 | 134.8 | 33.0 |
| P18C11_S179 | 51246 | 817 | 4418 | 92.7 | 43.7 | 136.1 | 32.9 |
| P18C12_S187 | 17674 | 448 | 3945 | 93.0 | 36.6 | 131.1 | 33.0 |
| P18D11_S180 | 21475 | 526 | 14998 | 96.7 | 138.2 | 134.6 | 33.2 |
| P18D12_S188 | 20357 | 2058 | 110291 | 99.9 | 999.0 | 136.0 | 33.0 |
| P18E11_S181 | 57620 | 827 | 10232 | 96.2 | 95.5 | 135.4 | 33.0 |
| P18E12_S1S9 | 31848 | 1295 | 55843 | 99.7 | 504.1 | 135.3 | 33.1 |
| P18F11_S182 | 19525 | 868 | 30185 | 98.4 | 277.5 | 135.9 | 33.1 |
| P18F12_S190 | 102572 | 1001 | 3061 | 98.3 | 28.7 | 137.2 | 32.6 |
| P18G11_S183 | 15274 | 473 | 8446 | 92.4 | 81.6 | 134.4 | 33.0 |
| P18H11_S184 | 14657 | 588 | 17551 | 93.6 | 168.1 | 134.6 | 32.9 |
| P18G12_S191 | 7327 | 4717 | 313457 | 99.9 | 2866.7 | 136.8 | 33.0 |

TABLE 5
Deconvolution results for variant strain detection, for alleles >1% of total raw reads.

| | | | | | % of | |
|---|---|---|---|---|---|---|
| | | | % REF | % ALT | Total | Adjusted |
| Position | Mutation | Present in Clades (Lineages) | alleles | alleles | Reads | Prevalence |
| 1059 | Orf1a | 20C, 20G, 20H (Beta-SA, V2), | 12% | 88% | 7.5% | 6.5% |
| | C1059T | 21C (Epsilon-CA), 21F (Iota-NY) | | | | |
| 5986 | Orf1a | 20I (Alpha-UK) | 43% | 57% | 1.4% | 0.8% |
| | C5986T | | | | | |
| 15279 | Orf1b | 20I (Alpha-UK) | 36% | 64% | 1.5% | 1.0% |
| | C15279T | | | | | |
| 25517 | Orf3a | 2IF (Iota-NY) | 8% | 92% | 6.2% | 5.7% |
| | C25517T | | | | | |
| 25563 | Orf3a | 20G, 20H (Beta-SA, V2), 21C | 7% | 93% | 5.5% | 5.1% |
| | Q57H | (Epsilon-CA), 21F (Iota-NY) | | | | |
| 28048 | Orf8 | 20I (Alpha-UK) | 52% | 48% | 3.2% | 1.6% |
| | R52I | | | | | |
| 28977 | N S235F | 20I (Alpha-UK) | 41% | 59% | 1.3% | 0.8% |
| | | Mean adjusted prevalence of 21F | 5.8% | | | |
| | | (Iota-NY) in wastewater | | | | |
| | | composite: | | | | |
| | | Mean adjusted prevalence of 20I | 1.0% | | | |
| | | (Alpha-UK) in wastewater | | | | |
| | | composite: | | | | |

% REF indicates the percentage of reads covering this allele that match the Wuhan reference sequence, % ALT indicates the percentage of reads covering the same allele that match a variant base sequence, % of Total Reads indicates the percent of the combined coverage of each allele out of the total mapped reads in the sample, Adjusted Prevalence is the produce of the % ALT and % of Total Reads.

The entire disclosure of all applications, patents, and publications cited herein are herein incorporated by reference in their entirety.

What is claimed:

1. A method of detecting and quantifying a virus-of-interest from human viral shed in a community, comprising:
concentrating viral material from a wastewater sample to form a viral nucleic acid material;
measuring an amount of a virus-of-interest nucleic acid in the viral nucleic acid material to obtain a first amount;
measuring an amount of a bacteriophage nucleic acid in the viral nucleic acid material to obtain a second amount; and
forming a ratio of the first amount to the second amount to estimate a level of virus-of-interest from human viral shed within a community.

2. The method of claim 1, wherein the virus-of-interest is an orthomyxovirus, influenza virus, influenza A, influenza B, an RNA virus, or a DNA virus.

3. The method of claim 2, wherein the RNA virus is characterized as one of Reoviridae, Picornaviridae, Caliciviridae, Togaviridae, Arenaviridae, Retroviridae, Flaviviridae, Orthomyxoviridae, Paramyxoviridae, Bunyaviridae, Rhabdoviridae, Filoviridae, Coronaviridae, Astroviridae, or Bornaviridae.

4. The method of claim 2, wherein the DNA virus is characterized as Adenoviridae, Papovaviridae, Parvoviridae, Herpesviridae, Poxviridae, or Hepadnaviridae.

5. The method of claim 1, wherein the virus-of-interest is SARS-CoV-2 or a SARS-CoV-2 variant.

6. The method of claim 1, wherein the bacteriophage nucleic acid is cross-assembly phage nucleic acid.

7. The method of claim 1, wherein concentrating viral material further comprises centrifuging the viral material through a sugar cushion in an ultracentrifuge.

8. The method of claim 1, wherein concentrating viral material further comprises centrifuging the viral material through a sugar gradient in an ultracentrifuge.

9. The method of claim 7, wherein centrifuging the viral material through a sugar cushion in an ultracentrifuge further comprises forming a solution comprising a buffer and sugar.

10. The method of claim 9, wherein the solution comprises a sugar in the amount of 40 to 60 percent weight of the total solution.

11. The method of claim 10, wherein the sugar comprises sucrose in an amount of about 50 percent weight of the total solution.

12. The method of claim 1, wherein the viral material comprises inactivated or fragmented virus.

13. The method of claim 1, wherein concentrating viral material from a wastewater sample to form a viral nucleic acid material is performed under conditions to purify the viral material.

14. The method of claim 1, wherein measuring the amount of virus-of-interest nucleic acid and bacteriophage nucleic acid is performed by qPCR analysis comprising one or more fluorescent materials.

15. A method of monitoring a virus-of-interest human transmission in a community, comprising:
(a) concentrating viral material from a wastewater sample to form a viral nucleic acid material;
(b) measuring an amount of virus-of-interest nucleic acid in the viral nucleic acid material to obtain a first amount;
(c) measuring an amount of bacteriophage nucleic acid in the viral nucleic acid material to obtain a second amount;
(d) forming a ratio of the first amount to the second amount to estimate a level of virus-of-interest human viral shed within a community; and
(e) repeating (a)-(d) in an amount sufficient to monitor virus-of-interest transmissions within a community.

16. The method of claim 15, wherein concentrating viral material further comprises centrifuging the viral material through a sugar cushion in an ultracentrifuge.

17. The method of claim 16, wherein centrifuging the viral material through a sugar cushion in an ultracentrifuge further comprises forming a solution comprising a buffer and sugar.

18. The method of claim 17, wherein the solution comprises a sugar in the amount of 40 to 60 percent weight of the total gradient-forming solution.

19. The method of claim 18, wherein the sugar comprises sucrose in an amount of about 50 percent weight of the total solution.

20. The method of claim 15, wherein the virus-of-interest is SARS-CoV-2.

21. A method of detecting and/or quantifying a virus-of-interest in wastewater, comprising:
concentrating viral material from a wastewater sample to form a viral nucleic acid material, wherein concentrating comprises centrifuging the viral material through a sugar cushion in an ultracentrifuge;
measuring an amount of virus-of-interest nucleic acid in the viral nucleic acid material to detect and/or quantify the virus-of-interest.

22. The method of claim 21, wherein centrifuging the viral material through a sugar cushion in an ultracentrifuge further comprises forming a solution comprising a buffer and sugar.

23. The method of claim 22, wherein the solution comprises a sugar in the amount of 40 to 60 percent weight of the total solution.

24. The method of claim 23, wherein the sugar comprises sucrose in an amount of about 50 percent weight of the total solution.

25. The method of claim 21, wherein the wastewater is characterized as an aliquot of less than 50 mL of raw wastewater.

26. The method of claim 21, wherein concentrating viral material comprises purifying the nucleic acid of the viral material.

27. The method of claim 21, wherein the virus-of-interest is SARS-CoV-2 or a SARS-CoV-2 variant.


SEQUENCE LISTING (14 sequences)

SEQ ID NO: 1 - DNA, 18 bp, artificial sequence
  Synthetic sequence
atgagcttagtcctgttg

SEQ ID NO: 2 - DNA, 18 bp, Artificial Sequence
  Synthetic sequence
ctccctttgttgtgttgt

SEQ ID NO: 3 - DNA, 21 bp, Artificial Sequence
  Synthetic sequence
agatgtcttgtgctgccggta

SEQ ID NO: 4 - DNA, 19 bp, Artificial Sequence
  Synthetic Sequence
ggtaactggtatgatttcg

SEQ ID NO: 5 - DNA, 20 bp, Artificial Sequence
  Synthetic sequence
ctggtcaaggttaatatagg

SEQ ID NO: 6 - DNA, 19 bp, Artificial Sequence
  synthetic sequence
tcatacaaaccacgccagg

SEQ ID NO: 7 - DNA, 30 bp, Artificial Sequence
  Synthetic Sequence
cagaagtacaaactcctaaaaaacgtagag

SEQ ID NO: 8 - DNA, 25 bp, Artificial Sequence
  Synthetic sequence
gatgaccaataaacaagccattagc

SEQ ID NO: 9 - DNA, 23 bp, Artificial Sequence
  Synthetic sequence
aataacgatttacgtgatgtaac

SEQ ID NO: 10 - DNA, 27 bp, Artificial Sequence
  synthetic sequence
caatgttacttggttccatgctatctc

SEQ ID NO: 11 - DNA, 29 bp, Artificial Sequence
  Synthetic sequence
ccagcctcttattatgttagacttctcag

SEQ ID NO: 12 - DNA, 35 bp, Artificial Sequence
  Synthetic sequence
ggtatcaaacctcttagtaccattggtcccagaga

SEQ ID NO: 13 - DNA, 151 bp, Artificial Sequence
  synthetic sequence
tacctttcttttccaatgttacttggttccatgctatctctgggaccaatggtactaagaggtttgataaccctgtcctaccatttaatgatggtgtttattttgcttccactgagaagtctaacataataagaggctggatttttggtac

SEQ ID NO: 14 - DNA, 157 bp, Artificial Sequence
  Synthetic sequence
tacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtac